(19) United States
(12) Patent Application Publication
(10) Pub. No.: US 2025/0019665 A1
(43) Pub. Date: Jan. 16, 2025

(54) SCALABLE BIOMANUFACTURING OF TUMOR ORGANOIDS

(71) Applicant: The Board of Trustees of The University of Alabama, Tuscaloosa, AL (US)

(72) Inventors: Alexandra DeVera Avera, Tuscaloosa, AL (US); Seungjo Park, Tuscaloosa, AL (US); Yonghyun Kim, Tuscaloosa, AL (US)

(21) Appl. No.: 18/772,579

(22) Filed: Jul. 15, 2024

Related U.S. Application Data
(60) Provisional application No. 63/526,721, filed on Jul. 14, 2023.

(51) Int. Cl. C12N 5/071; C12N 5/079; C12N 5/09

(52) U.S. Cl. CPC C12N 5/0697; C12N 5/0618; C12N 5/0693; C12N 2501/11; C12N 2501/115; C12N 2513/00

(57) ABSTRACT

Disclosed herein are matrix-free and in vivo-like glioblastoma organoids (GBOs) developed using patient-derived xenograft GBM lines in small-scale bioreactors and a method of scalable biomanufacture thereof. Shear stress, agitation rate, and media supplements can be optimized to produce highly reproducible GBOs over 1 mm diameter within 4-5 weeks. GBOs can exhibit high stemness and strong cell-to-cell interactions compared to conventional tumorsphere cultures. They can display spatial gradients of HIF-1α positive hypoxic cores where CD133-positive cells resided and spatially heterogeneous expression of NOTCH and its ligands. A self-established, hierarchically organized, and heterogeneous TME by GBM transdifferentiation into endothelial cells, pericytes, and astrocytes can also be observed. Collectively, the ability to biomanufacture uniformly sized GBOs that recapitulate in vivo GBM TME features that can serve as an improved GBM in vitro model is demonstrated herein.

### CROSS REFERENCE TO RELATED APPLICATIONS

This application claims the benefit of priority to U.S. Provisional Application No. 63/526,721, filed Jul. 14, 2023, which is incorporated by reference herein in its entirety.

### STATEMENT OF GOVERNMENT SUPPORT

This invention was made with government support under Grant No. 2000053 awarded by the National Science Foundation and Grant No. P200A210069 awarded by the Department of Education. The government has certain rights in the invention.

### BACKGROUND

Glioblastoma (GBM) is the most common and malignant primary brain tumor with a poor prognosis (Ostrom et al., 2020). Due to its invasiveness and heterogeneity, GBM remains an almost incurable disease with existing therapies (van Linde et al., 2017). Glioblastoma stem cells (GSCs) are thought to be the origin of GBM and orchestrate the tumor microenvironment (TME) by way of suppressing immune responses, inducing angiogenesis, and differentiating into multi-lineage cells (Lathia et al., 2015). Many studies targeting GSCs have shown promising results and expanded understanding of GBM therapies (Dong et al., 2019; Emlet et al., 2014; Fan et al., 2010; Shah et al., 2019; Sherry et al., 2009; Vora et al., 2020). However, applications and reproducibility of these studies have been challenged due to hierarchically organized inter- and intratumoral heterogeneity of GBM. Development of GBM organoids (GBOs) retaining heterogeneity of GBM is crucial for reliable in vitro GBM models.

Patient-derived xenograft (PDX) GBM models have been appreciated as the gold standard preclinical models for the disease. The PDX model is developed by implanting dissociated primary GBM cells into animal models. These models preserve the heterogeneous GBM features and the interactions between GBM and innate surrounding brain tissues; however, variations in the orthotopic engraftment efficiency, low throughput, and long duration for tumorigenesis have been challenging (Patrizii et al., 2018). It typically takes two to 11 months to generate PDX models, which is simply too long given GBM patients' median survival is 15 months.

Three-dimensional (3D) GBM tumorspheres have been widely accepted as a good surrogate in vitro GBM model to the PDX models. The tumorspheres are spontaneously formed in stem cell-enriching media supplemented with basic fibroblast growth factor (bFGF) and epidermal growth factor (EGF), and they highly display stem cell-like characteristics and tumorigenesis capability (Lee et al., 2006; Zhu et al., 2011). The tumorsphere model retains a high GSC population inside the sphere, but it does not preserve cell-to-cell interactions between differentiated GBM and GSCs, which support the GSC niche and tumor growth (Wang et al., 2018). To improve upon the tumorspheres, GBM organoids (GBOs) were developed using Matrigels, together with the addition of Rho kinase (ROCK) inhibitor (ROCKi) and other mitogens (Hubert et al., 2016). In this system, GBOs were able to continuously grow up to one year and exhibit an oxygen gradient with hypoxia features. However, these GBOs were developed by aggregating different colonies in the Matrigel. Also, the growth of GBOs became stagnant after a few weeks.

Thus, there is a need for a means of producing GBOs at scale and quickly that can accurately replicate the tumor microenvironment. These needs and others are at least partially satisfied by the present disclosure.

### SUMMARY

Disclosed herein is a method for standardized, scalable biomanufacturing of tumor organoids based on defined engineering parameters and practices. The defined engineering parameters and practices include, but are not limited to, organoid size, cell culture media formulation, bioreactor vessel geometries, fluid shear stress, cleaning-in-place protocols (CIPs), and long-term storage of organoids in an organoid biobank. Organoids are in vitro millimeter-scale multi-cellular tissue models, often grown from individual stem cells. Tumor organoids utilize easily attainable and fast-growing cancer stem cells to remodel native patient tumor in vitro. Other tumor models include utilizing native tissue explants, induced pluripotent stem cells or embryonic stem cells, all of which are difficult to obtain and control for differentiation into the desired tissue. Tumor organoids have become the newest generation of cancer modeling by offering the benefits to easily observe cell-cell interactions, cell-to-matrix interactions, and cellular differentiation and organization into highly heterogeneous tumor microenvironments during tumor development. Tumor organoids are becoming increasingly popular in biological and biomedical research labs for pre-clinical in vitro testing without the limitations of accessibility from real tumors. However, generating and storing high quantities of tumor organoids is difficult because the process is not standardized and is highly laborious.

Disclosed herein are matrix-free and in vivo-like GBM organoids (GBOs) developed using patient-derived xenograft GBM lines in small-scale biorcactors and a method of scalable bio-manufacture thereof. Shear stress, agitation rate, and media supplements can be optimized to produce highly reproducible GBOs over 1 mm diameter within 4-5 weeks. GBOs can exhibit high stemness and strong cell-to-cell interactions compared to conventional tumorsphere cultures. They can display spatial gradients of HIF-1α positive hypoxic cores where CD133-positive cells resided and spatially heterogeneous expression of NOTCH and its ligands. A self-established, hierarchically organized, and heterogeneous TME by GBM transdifferentiation into endothelial cells, pericytes, and astrocytes can also be observed. Collectively, the ability to biomanufacture uniformly sized GBOs that recapitulate in vivo GBM TME features that can serve as an improved GBM in vitro model is demonstrated herein.

In some aspects, the techniques described herein relate to a method of biomanufacturing organoids including inducing disposing a cellular starting material in a bioreactor and continuously exposing the cellular starting material to mechanical stress.

In some aspects, the techniques described herein relate to a tumor model including an organoid, wherein the organoid is derived from human stem cells.

### BRIEF DESCRIPTION OF DRAWINGS

FIG. 1 depicts a proposed scalable tumor organoid biomanufacturing process.

FIG. 2 depicts organoid samples grown with the proposed scalable tumor organoid biomanufacturing process described in FIG. 1.

FIG. 3A-3B depict organoid diameter and circularity during culture, where the organoids are grown with the proposed scalable tumor organoid biomanufacturing process described in FIG. 1.

FIGS. 4A-4B depict glioblastoma organoid tissue grown with the proposed scalable tumor organoid biomanufacturing process (FIGS. 4A-4B).

FIGS. 5A-5F depict optimization of glioblastoma organoid (GBO) production. FIG. 5A shows cell number fold change after 5 days culture in two different bioreactors (BR1: D/d 1.5; BR2: D/d 1.3) at 60 rpm. P<0.05, n≥3. FIG. 5B shows brightfield images of GBO. Scale bar: 100 μm. FIG. 5C shows continuous U87 culture in the BR2 configuration. FIG. 5D shows inhibition of cell proliferation by Y-27632 Rho-associated protein kinase inhibitor (ROCKi). Cell number after 5 days of culture in the BR2 configuration with 60 and 120 rpm agitation was normalized to the initial seeding number. FIG. 5E shows brightfield images of GBOs in BR2 with 60 and 120 rpm and with and without ROCKi. Scale bar: 100 μm.

FIG. 5F shows perspective views of live and dead cells in GBO. Main square: top view; left panel: side view; and bottom panel: front view. Green: live cells, red: apoptotic cells.

FIGS. 6A-6B depict optimization of glioblastoma organoid (GBO) production. FIG. 6A shows immunohistochemistry (IHC) of GBO stained for SOX2 in green and CD44 in red. Nucleus in blue. Scale bar: 100 μm. FIG. 6B shows quantified fluorescence intensity of SOX2 and CD44 by CTCF; n>10; mean±SD.

FIGS. 7A-7C depict production of large GBOs within 5 weeks. FIG. 7A shows U251 and JX6 GBOs generated from the bioreactors (120 rpm; bGBO) and tissue culture polystyrene (0 rpm; tGBO). Culture period is indicated above the images by week. Scale bar: 100 μm. FIG. 7B shows growth curves of JX6 (diamonds) and U251 (triangles) bGBOs. Solid line: live cell number, dotted line: cell viability. N>3; mean±SD. FIG. 7C shows a principal component analysis (PCA) plot of diameter and circularity for the JX6 (blue) and U251 (red) tGBOs (0 rpm) and bGBOs (120 rpm) with 90% confidence ellipses (light blue: 0 rpm, light red: 120 rpm). ∘: 0 rpm and +: 120 rpm.

FIGS. 8A-8D depict production of large GBOs within 5 weeks. FIGS. 8A-8B show box and whisker plot of the size distribution of JX6 (FIG. 8A) and U251 (FIG. 8B) tGBOs (0 rpm) and bGBOs (120 rpm). FIGS. 8C-8D show box and whisker plots of the circularity of JX6 (FIG. 8C) and U251 (FIG. 8D) tGBOs (0 rpm) and bGBOs (120 rpm).

FIGS. 9A-9B depict SEM and TEM image analyses of tGBOs and bGBOs and their extracellular (EVs). FIGS. 9A-9B shows SEM images of tGBOs (0 rpm) (FIG. 9A) and bGBOs (120 rpm) (FIG. 9B). Arrows indicate: axon-like long protrusion (second row, second and third columns); strong cell-to-cell interactions (first row, fourth column); and tight junctions (third row, third and fourth columns). FIGS. 9A-9B also show average diameters of GBOs indicated on the left side. Scale bars from left to right columns: 100 μm, 50 μm, 10 μm, and 2 μm.

FIGS. 10A-10C depict SEM and TEM image analyses of tGBOs and bGBOs and their extracellular (EVs). FIG. 10A shows SEM images of Evs on the tGBO and bGBO. (i) tGBO, (ii) bGBO, and (iii) bGBO with an axon-like long protrusion. Broken-line boxes: higher magnification images in the second and third rows also showing arrows: budding EVs; third row, first and third column arrows: exosomes; third row, second column arrows: larger size exosomes than those in (i) and (iii). Scale bars top to bottom rows: 20 nm, 2 nm, and 1 nm. FIG. 10B shows a high-density region (HDR) plot for the size of EVs and exosomes. Dark boxes represent the 50% highest cumulative of the total population. Lighter boxes represent 99% cumulative probability of the total population. The upper point represents an outlier with 99% probability. The mode lines are indicated. *p<0.05. FIG. 10C shows TEM images of exosomes secreted in the conditioned media of tGBO and bGBO cultures collected after 14 days. Scale bars: 100 nm.

FIGS. 11A-11B depict multivariate analysis of gene expression in U251 bGBOs. FIG. 11A shows a scatter plot matrix of gene expression and the bGBO size (white boxes) and the corresponding Pearson correlation coefficients (shaded heatmap boxes). The y-axis of the top left corner box and the x-axis of the bottom left corner box are the size (diameter) of the bGBOs in μm. Other boxes' axes indicate relative gene expression. FIG. 11B shows a heatmap of Pearson correlations (left) and their probability p-values (right) between the size and the molecular profiles of heterogeneous GBOs.

FIGS. 12A-12B depict multivariate analysis of gene expression in U251 bGBOs. FIG. 12A shows a cell plot of gene expression level by each week of culture (W) with normalized gene expression (Z) to the Day I gene expression. FIG. 12B shows a principal components analysis (PCA) plot of U251 gene expression for different sizes of the bGBOs with 90% confidence ellipses (shaded ellipses). Lower left quadrant: bGBO<400 μm; upper left quadrant: 400 μm<bGBO<700 μm; lower right quadrant: bGBO>700 μm.

FIG. 13 depicts relative expression levels of genes (y-axis) in U251 bGBOs by the size of bGBO (x-axis). R² values are in the table below the graphs.

FIG. 14 depicts predictive gene expression profiles in U251 bGBOs according to the size of the bGBO and relative gene expression of HIF-1α, NOTCH1, and NOTCH2. The x-axes indicate the bGBO size in μm and relative gene expression for each gene. 95% confidence interval are shown. Dotted lines indicate the predicted size and gene expression of HIF-1α, NOTCH1, and NOTCH2.

FIGS. 15A-15C depict a size-dependent heterogeneous glioblastoma stem cell (GSC) niche model in JX6 PDX GBOs. FIG. 15A shows IHC of different sizes of JX6 bGBOs, with higher magnification of images in the bottom rows. FIG. 15B shows IHC of uGBOs, tGBOs, and DGCs. FIGS. 15A-15B show IHC of GBO stained for GSC marker: CD133 (green) and hypoxic marker: HIF-1α (red). FIG. 15C shows IHC of bGBOs for CD133 (green) and endothelial cell marker: CD31 (purple). Nuclei (blue). Scale bar: 100 μm.

FIGS. 16A-16C depict a size-dependent heterogeneous glioblastoma stem cell (GSC) niche model in JX6 PDX GBOs. FIG. 16B shows IHC of different sizes of JX6 bGBOs, with higher magnification of images in the bottom rows. FIG. 16C shows IHC of uGBOs, tGBOs, and DGCs. FIG. 16A shows IHC of bGBOs stained for GSC markers: CD133 (green) and CD44 (red). FIGS. 16B-16C show IHC of GBOs stained for GSC marker: NOTCH1 (green) and NOTCH ligand: DLL1 (red). Nuclei (blue). Scale bar: 100 μm.

FIGS. 17A-17D depict a size-dependent heterogeneous glioblastoma stem cell (GSC) niche model in JX6 PDX GBOs. FIG. 17B and FIG. 17C show IHC of different sizes of JX6 bGBOs, with higher magnification of images in the bottom rows. FIG. 17D shows IHC of uGBOs, tGBOs, and DGCs. FIG. 17A shows IHC of bGBO stained for NOTCH1 (green) and NOTCH ligand: DLL4 (red). FIG. 17B and FIG. 17D show IHC of GBO stained for angiogenesis marker: VEGFR 1 (green) and endothelial cell marker: CD144 (red). FIG. 17C shows IHC of bGBO stained for pericyte marker: CD146 (green), CD144 (red), and astrocyte marker: MAP2 (purple). Nuclei (blue). Scale bar: 100 μm.

FIGS. 18A-18D depict transdifferentiated GBM and their spatial distribution in JX6 GBOs. FIG. 18A and FIG. 18C show IHC of different size of JX6 bGBOs with higher magnification images in the bottom rows. FIG. 18B and FIG. 18D show IHC of uGBOs, tGBOs, and JX6 DGCs. FIGS. 18A-18B show a pericyte marker: α-SMA (green), endothelial cell marker: CD34 (red), and astrocyte marker: GFAP (purple). FIGS. 18C-18D show an astrocyte marker: S100B (green) and pericyte marker: NG2 (red). (c) and (f) CD146 (green), CD144 (red), and MAP2 (purple). Nuclei (blue). Scale bar: 100 μm.

FIGS. 19A-19B depict transdifferentiated GBM and their spatial distribution in JX6 GBOs. FIG. 19A shows IHC of different size of JX6 bGBOs with higher magnification images in the bottom rows. FIG. 19B shows IHC of uGBOs, tGBOs, and JX6 DGCs. Nuclei (blue). Scale bar: 100 μm.

FIGS. 20A-20B depict a GBM TME containing a necrotic core. FIGS. 20A-20B show IHC of JX6 bGBO with higher magnification images of the indicated regions in the bottom two rows. FIG. 20A shows VEGFR1 (green), CD144 (red), and CD31 (purple). FIG. 20B shows α-SMA (green), CD34 (red), and GFAP (purple). Nuclei (blue). NC: necrotic core, VM: vasculogenic mimicry, HN: hypoxic niche, and PVN: perivascular niche. Scale bar: 100 μm.

FIGS. 21A-21B depict a GBM TME containing a necrotic core. FIGS. 21A-21B show IHC of JX6 bGBO with higher magnification images of the indicated regions in the bottom two rows. FIG. 21A shows S100B (green) and NG2 (red). FIG. 21B shows CD133 (green), CD31 (red), and MAP2 (purple). Arrows indicate a wrinkled artifact on the sectioned bGBO. Nuclei (blue). NC: necrotic core, VM: vasculogenic mimicry, HN: hypoxic niche, and PVN: perivascular niche. Scale bar: 100 μm.

FIGS. 22A-22B depict a GBM TME containing a necrotic core. FIG. 22A shows IHC of JX6 bGBO with higher magnification images of the indicated regions in the bottom two rows. FIG. 22A shows NOTCH1 (green) and DLL1 (red). Arrows indicate a defect during the sample preparation. Nuclei (blue). NC: necrotic core, VM: vasculogenic mimicry, HN: hypoxic niche, and PVN: perivascular niche. Scale bar: 100 μm. FIG. 22B shows the quantified intensity of NOTCH1 and DLL1 in HN and PVN by CTCF. *p<0.05, n>10; mean±SD.

FIGS. 23A-23B depict a spatially and hierarchically organized GSC hypoxic niche. FIGS. 23A-23B show a JX6 bGBO model having necrotic TME. FIG. 23B shows higher magnification images of indicated regions in the bottom two rows. FIGS. 23A-23B show a tight junction marker: ZO1 (green). Nuclei (blue). NC: necrotic core, HN: hypoxic niche, and PVN: perivascular niche. Scale bar: 100 μm.

FIGS. 24A-24B depict a spatially and hierarchically organized GSC hypoxic niche. FIGS. 24A-24B show a JX6 bGBO model having necrotic TME. Higher magnification images of indicated regions are shown in the bottom two rows. FIGS. 24A-24B show HIF-1a (red). In FIG. 24A, yellow arrows indicate bubble between sample and the cover slide. FIG. 24B shows a transcription factor: SALL2 (green). White arrows indicate the potential hypoxic niche. Nuclei (blue). NC: necrotic core, HN: hypoxic niche, and PVN: perivascular niche. Scale bar: 100 μm.

FIG. 25 depicts quantification and comparison of the tumor microenvironment in JX6 PDX GBOs. FIG. 25 shows normalized fluorescent intensity of target protein per cell as a function of position (i.e., normalized radial distance) of an intensity profile vector spanning the whole diameter of uGBO, tGBO, bGBO<500 μm, and bGBO>800 μm. FIG. 25 shows a GSC marker: CD133 (green) and hypoxic marker: HIF-1α (red) for size-dependent heterogeneous GSC niche. All data presented as mean±SE. *p<0.05, ***p<0.001, ****p<0.0001. α-SMA, α-smooth muscle actin; bGBO, GBOs produced from bioreactors; GBM, glioblastoma; GBO, GBM organoid; GFAP, glial fibrillary acidic protein; GSC, glioblastoma stem cell; HIF-1α, hypoxia-inducible factor 1α; PDX, patient-derived xenograft; tGBO, GBOs produced from static tissue culture flasks; TME, tumor microenvironment; uGBO, GBOs produced from U-bottom well plates.

FIG. 26 depicts quantification and comparison of the tumor microenvironment in JX6 PDX GBOs. FIG. 26 shows normalized fluorescent intensity of target protein per cell as a function of position (i.e., normalized radial distance) of an intensity profile vector spanning the whole diameter of uGBO, GBO, bGBO<500 μm, and bGBO>800 μm. FIG. 26 shows a pericyte marker: α-SMA (green), endothelial cell marker: CD34 (red), and astrocyte marker: GFAP (magenta) for transdifferentiated GBM cells. All data presented as mean±SE. *p<0.05, ***p<0.001, ****p<0.0001. α-SMA, α-smooth muscle actin; bGBO, GBOs produced from biorcactors; GBM, glioblastoma; GBO, GBM organoid; GFAP, glial fibrillary acidic protein; GSC, glioblastoma stem cell; HIF-1α, hypoxia-inducible factor 1α; PDX, patient-derived xenograft; tGBO, GBOs produced from static tissue culture flasks; TME, tumor microenvironment; uGBO, GBOs produced from U-bottom well plates.

FIG. 27 shows normalized fluorescent intensity of markers versus uGBO, tGBO, bGBO<500 μm, and bGBO>800 μm in the core (top row) and the periphery (bottom row) of each GBO sample stained for GSC marker: CD133, hypoxic marker: HIF-1α, pericyte marker: α-SMA, endothelial cell marker: CD34, and astrocyte marker: GFAP. All data presented as mean±SE. *p<0.05, ***p<0.001, ****p<0.0001. α-SMA, α-smooth muscle actin; bGBO, GBOs produced from bioreactors; GBM, glioblastoma; GBO, GBM organoid; GFAP, glial fibrillary acidic protein; GSC, glioblastoma stem cell; HIF-1α, hypoxia-inducible factor 1α; PDX, patient-derived xenograft; tGBO, GBOs produced from static tissue culture flasks; TME, tumor microenvironment; uGBO, GBOs produced from U-bottom well plates.

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s) but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, “multipotent” cells are more differentiated than pluripotent cells but are not permanently committed to a specific cell type. Pluripotent cells therefore have a higher potency than multipotent cells.

As used herein, “pluripotent cells” means a population of cells capable of differentiating into all three germ layers and becoming any cell type in the body. Pluripotent cells express a variety of cell surface markers, have a cell morphology characteristic of undifferentiated cells and form teratomas when introduced into an immunocompromised animal, such as a SCID mouse. Teratomas typically contain cells or tissues characteristic of all three germ layers.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell include, without limitation, a progenitor nerve cell.

As used herein, “serum-free” means that neither the culture nor the culture medium contains serum or plasma, although purified or synthetic serum or plasma components (e.g., FGFs) can be provided in the culture in reproducible amounts as described below.

As used herein, “stem cell” defines an adult undifferentiated cell that can produce itself and a further differentiated progeny cell and under certain situations, give rise to specialized cells.

As used here, a “substantially pure population” means a population of derived mesenchymal cells that contains at least 99% mesenchymal cells. Cell purification can be accomplished by any means known to one of ordinary skill in the art. For example, a substantially pure population of cells can be achieved by growth of cells or by selection from a less pure population, as described herein.

To facilitate an understanding of the principles and features of various embodiments of the present invention, they are explained hereinafter with reference to their implementation in illustrative embodiments.

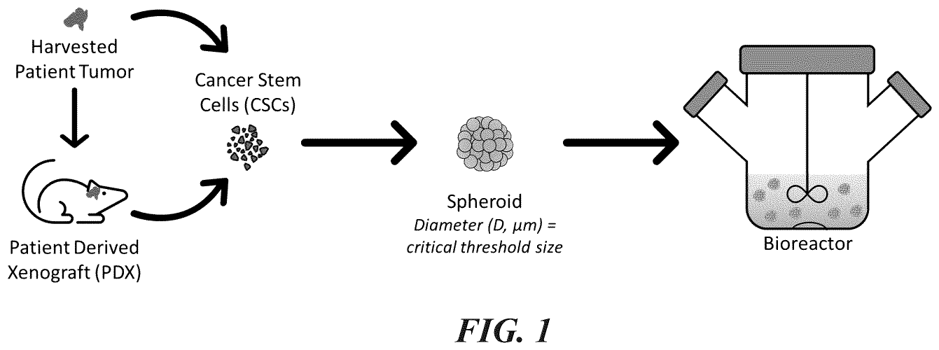

In an implementation, a bio-manufacturing method to produce organoids is described. A cellular starting material is disposed in a bioreactor and continuously exposed to mechanical stress. The method is shown in FIG. 1, wherein precursor cells are harvested from a source, such as cancer stem cells or patient derived Xenograft (PDX).

In some aspects, the cellular starting material is precursor cells. In some such aspects, spheroid formation from the precursor cells is induced by the continuous mechanical stress in the bioreactor. A spheroid of the precursor cells is induced to grow until a critical threshold size. The spheroid is formed without the use of a scaffolding matrix, such as commonly used Matrigel, or induced differentiation. The critical threshold size is determined when the spheroid responds to its physical environment and exhibits self-differentiation.

The critical threshold size may be from about 100 μm to about 600 μm. In particular, the critical threshold size may be about 150 μm, about 200 μm, about 250 μm, about 300 μm, about 350 μm, about 400 μm, about 450 μm, about 500 μm, or about 550 μm.

In other aspects, the cellular starting material is spheroids, which may be obtained by any known method. Once the spheroids reach the critical threshold size, a plurality of spheroids are inoculated in a bioreactor and exposed to continuous mechanical stress. The critical threshold size is determined when the spheroid responds to its physical environment and exhibits self-differentiation.

The critical threshold size may be from about 100 μm to about 600 μm. In particular, the critical threshold size may be about 150 μm, about 200 μm, about 250 μm, about 300 μm, about 350 μm, about 400 μm, about 450 μm, about 500 μm, or about 550 μm.

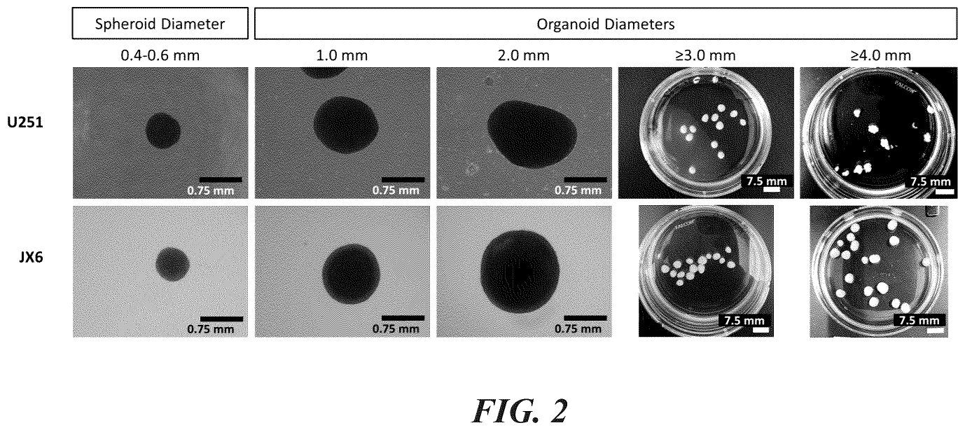

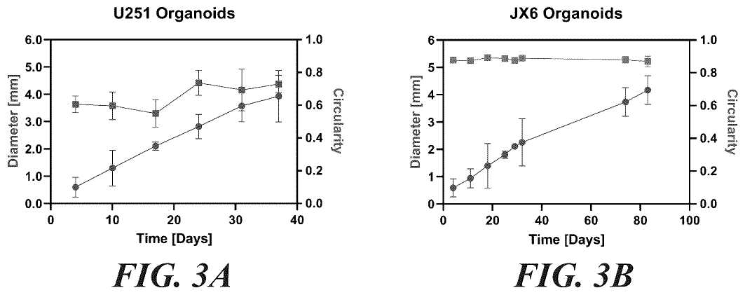

In some aspects, the method may be applied to batch processing of precursor cells or spheroids to obtain reproducible, spherical organoids, as shown in FIG. 2 and FIGS. 3A-3B. In some such aspects, a size of the bioreactor may be chosen for a desired batch size (i.e., number of product organoids).

The mechanical stress may be shear stress and/or agitation. In one aspect, the mechanical stress is shear stress and agitation. In some implementations, the mechanical stress may be achieved by use of magnetic stirring and/or stirring with a baffle having a single blade or multiple blades. In one implementation, the mechanical stress is achieved by the use of magnetic stirring and a single-blade baffle.

In some aspects, the cellular starting material is exposed to shear stress from about 0.1 Pa to about 0.7 Pa. In particular, the cellular starting material is exposed to shear stress of about 0.2 Pa, about 0.3 Pa, about 0.4 Pa, about 0.5 Pa, or about 0.6 Pa.

In some aspects, the cellular starting material is exposed to agitation having a rate of about 60 rpm to about 120 rpm. In particular, the cellular starting material is exposed to agitation having a rate of about 70 rpm, about 80 rpm, about 90 rpm, about 100 rpm, or about 110 rpm.

In some implementations, the bioreactor has a Reynolds number of about 600 to about 2200. In particular, the bioreactor has a Reynolds number of about 800, about 1000, about 1200, about 1400, about 1600, about 1800, or about 2000.

The organoids are allowed to grow in the bioreactor until the organoids reach a diameter of at least 0.5 mm. The mechanical stress is continuously applied in the bioreactor until the desired organoid diameter is obtained. The organoids may be transferred to a new bioreactor following clean-in-place protocols in order to prevent fouling. During organoid growth in the bioreactor, care is taken not to overcrowd the bioreactor.

In some aspects, the produced organoids may have a diameter of about 500 μm to about 5 mm. In particular, the produced organoids have a diameter of about 600 μm, about 700 μm, about 800 μm, about 900 μm, about 1 mm, about 1.5 mm, about 2 mm, about 2.5 mm, about 3 mm, about 3.5 mm, about 4 mm, or about 4.5 mm.

In some implementations of the disclosed method, an organoid having a diameter of about 4 mm may be grown in the bioreactor in about 6 weeks. In other implementations, the organoid having a diameter of about 4 mm may be grown in about 5 weeks, about 7 weeks, or about 8 weeks. In yet other aspects, an organoid having a diameter of about 2 mm may be grown in about 3 weeks, about 4 weeks, or about 5 weeks. For example, an organoid may grow to a diameter in a number of days as shown in FIGS. 3A-3B.

In some implementations, the bioreactor size and geometry, applied shear stress, and rate of agitation are chosen to produce an organoid with a desired diameter. Examples of bioreactor systems resulting in pre-determined organoid size are given in the Examples attached hereto.

In some implementations, the cellular starting material is or is derived from human stem cells. In some particular implementations, the cellular starting material is or is derived from cancer stem cells such as glioblastoma stem cells, breast cancer stem cells, skin tissue stem cells, or any other cancer stem cell types of clinical relevance. In some aspects, when the cellular starting material is or is derived from glioblastoma stem cells, the produced organoids comprise glioblastoma organoids. In other aspects, when the cellular starting material is or is derived from breast cancer stem cells, the produced organoids comprise breast cancer organoids. In other particular implementations, the cellular starting material is or is derived from healthy stem cells such as neural progenitor cells. In some aspects, when the cellular starting material is or is derived from neural progenitor cells, the produced organoids comprise healthy neural organoids.

In other implementations, the precursor cells are in a cell media formulation chosen for the desired organoid type and size. The cell culture media is chosen to retain the stemness of the precursor cells. In yet other implementations, cleaning-in-place protocols are used to affect the desired organoid. In some implementations, the organoids may be stored, in particular, the organoids may be cryogenically stored.

In an implementation, a matrix-free tumor model is disclosed. The matrix-free tumor model comprises an organoid, in particular, a glioblastoma organoid. The glioblastoma organoid (GBO) may comprise a hypoxic niche and/or a perivascular niche. In one aspect, the GBO comprises a hypoxic niche and a perivascular niche.

In some aspects, the GBO is stable under shear stress of about 0.1 Pa to about 0.7 Pa. In particular, the GBO is stable under shear stress of about 0.2 Pa, about 0.3 Pa, about 0.4 Pa, about 0.5 Pa, or about 0.6 Pa. In other aspects, the GBO is stable under agitation having a rate of about 60 rpm to about 120 rpm. In particular, the GBO is stable under agitation having a rate of about 70 rpm, about 80 rpm, about 90 rpm, about 100 rpm, or about 110 rpm.

In some aspects, the GBO may have a diameter of about 500 μm to about 5 mm. In particular, the produced organoids have a diameter of about 600 μm, about 700 μm, about 800 μm, about 900 μm, about 1 mm, about 1.5 mm, about 2 mm, about 2.5 mm, about 3 mm, about 3.5 mm, about 4 mm, or about 4.5 mm.

In yet other aspects, the tumor model comprises axon-like protrusions.

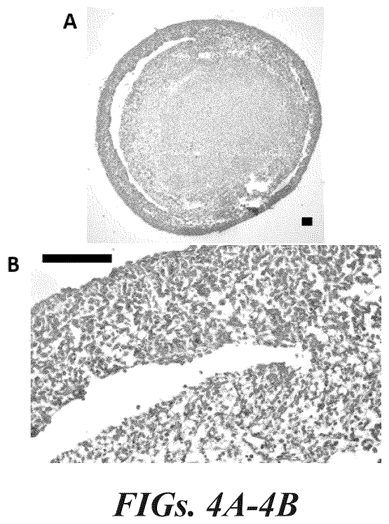

In one example of the disclosed method and tumor model, the tumor model (FIGS. 4A-4B) and a previously published tumor organoid model (Sundar, 2022) are shown with contrasting dyes in standard pathology tests. The previously published tumor model was developed over three months from a kit containing Matrigel and induced differentiation reagents. The disclosed organoid model was developed at a fraction of cost and time and is easily transported after development.

### EXAMPLES

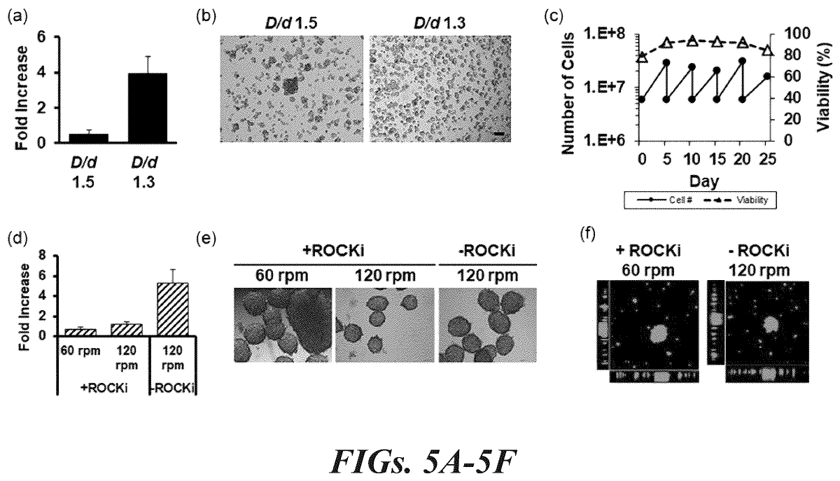

Example #1—Optimization of GBM Organoids (GBOs) Production: Various bioreactor vessel configurations and parameters were tested to optimize GBO culture conditions (Table 1, Table 2). Operating parameters were selected to maintain maximum shear stresses from 0.2 Pa to 0.4 Pa, which was previously reported to minimize shear-induced cell death (Panchalingam et al., 2010). First, different bioreactor geometries were tested: vessel diameter (D) over impeller diameter (d) with the same shear stress (0.2 Pa). The BR2 (D/d 1.3) promoted significantly better cell proliferation compared to the BR1 (D/d 1.5) and produced uniformly sized (FIGS. 5A-5B). In the BR2 condition, the cell proliferation was reproducible, and the cell viability remained high (FIG. 5C). With the BR2 geometries, the higher agitation rate (120 rpm) slightly improved cell proliferation (FIG. 5D).

TABLE 1
Vessel geometries and impeller diameters
of two small-scale bioreactors (BR)

| Parameters | BR1 | BR2 |
|---|---|---|
| b (cm) | 1.5 | 2.5 |
| C (cm) | 1 | 1 |
| D (cm) | 6 | 6.5 |
| d (cm) | 4 | 5 |
| D/d | 1.5 | 1.3 |
| n_{B} | 0 | 0 |
| nₚ | 2 | 2 |
| ρ (kg/m³) | 1008 | — |
| υ (m²/s) | 8.43 × 10⁻⁷ | — |

TABLE 2
Vessel geometries and impeller diameters
of two small-scale bioreactors (BR)

| Parameters | BR1: D/d 1.5 | | BR2: D/d 1.3 | |
|---|---|---|---|---|
| V_{w} (ml) | 60 | 125 | 60 | 60 |
| H (cm) | 3 | 5 | 2.5 | 2.5 |
| C/H | 0.33 | 0.2 | 0.4 | 0.4 |
| N (rpm) | 60 | 120 | 60 | 60 |
| Re_{G} | 816.14 | 1632.29 | 792.68 | 1585.37 |
| N_{P0} | 0.68 | 0.88 | 0.36 | 0.28 |
| P (W) | 6.98 × 10⁻⁵ | 7.27 × 10⁻⁵ | 1.14 × 10⁻⁴ | 7.06 × 10⁻⁴ |
| P/V_{L} (W m⁻³) | 1.55 | 5.81 | 1.91 | 11.77 |
| ε (m² s⁻³) | 1.15 × 10⁻³ | 5.77 × 10⁻³ | 1.89 × 10⁻³ | 1.17 × 10⁻² |
| νₜᵢₚ (m s⁻¹) | 0.13 | 0.25 | 0.16 | 0.31 |
| γ (μm) | 26.80 | 17.92 | 23.68 | 15.02 |
| τₘₐₓ (Pa) | 0.19 | 0.38 | 0.21 | 0.53 |

*Bioreactor culture consider are presented in SI units. Modified Reynold's number (Re_{G}), power number (N_{P0}), power input (P), disipated for power mass (ε), tip speed, eddies length, and maximum shear stress (τₘₐₓ) were calculated in Kamei correlation (Furukawa et al., 2012)

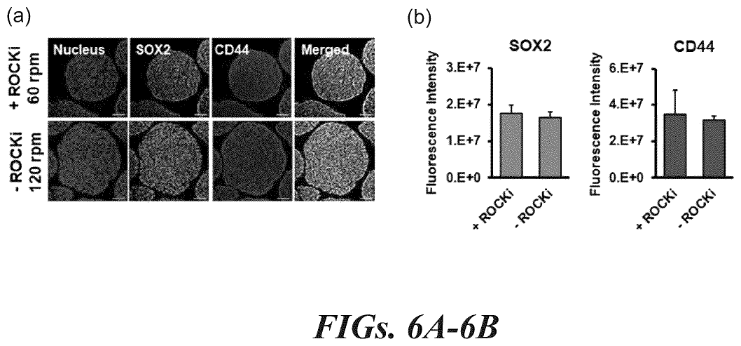

Next, the effects of Rho-associated protein kinase inhibitor (ROCKi) Y-27632 on different bioreactor culture conditions were tested. It was previously reported to enhance GBM tumorsphere formation and expansion of pluripotent stem cells (Liu et al., 2014; Tilson et al., 2015). To verify the effects, U251 was inoculated as single cells with or without ROCKi. Cells grown without ROCKi showed significantly increased cell proliferation compared to those with ROCKi (FIG. 5E). To test whether low cell proliferation is a result of apoptosis, live and dead cells were stained. ROCKi did not induce apoptosis in GBO (FIG. 6A). Regardless of ROCKi, the higher agitation rate (120 rpm) induced significantly higher cell proliferation compared to the lower agitation rate (60 rpm) and produced more uniformly sized GBOs (FIGS. 5E-5F). This was more pronounced in the BR2 condition (D/d=1.3), where the resulting power input was nearly 7-fold higher due to the higher agitation rate (Table 2). Although the BR2 with the lower agitation rate (60 rpm) did generate GBOs greater than 500 μm in diameter, these organoids were not uniform in shape and were more likely formed by random aggregations (FIG. 5F).

To further examine whether ROCKi maintained GSC stemness, the expression of GSC markers, SOX2 and CD44, were analyzed (FIG. 6B). Regardless of the presence of ROCKi, SOX2 and CD44 were highly expressed, and their expression levels were not significantly different.

Collectively, the BR2 condition (D/d 1.3) with the high agitation rate (120 rpm) improved cell proliferation, prevented GBO production from random aggregations, and produced GBOs with less variation in their size. ROCKi inhibited cell proliferation in the bioreactor but did not induce apoptosis. Also, ROCKi did not alter GSC protein marker expression.

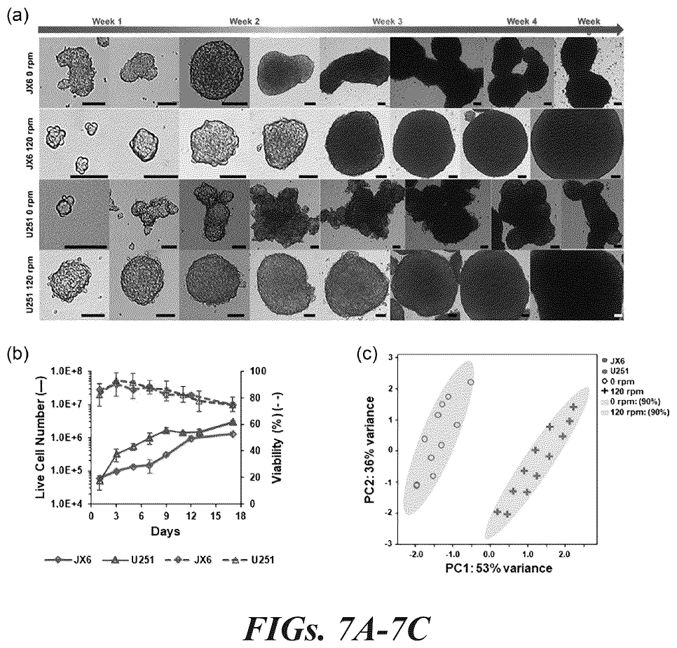

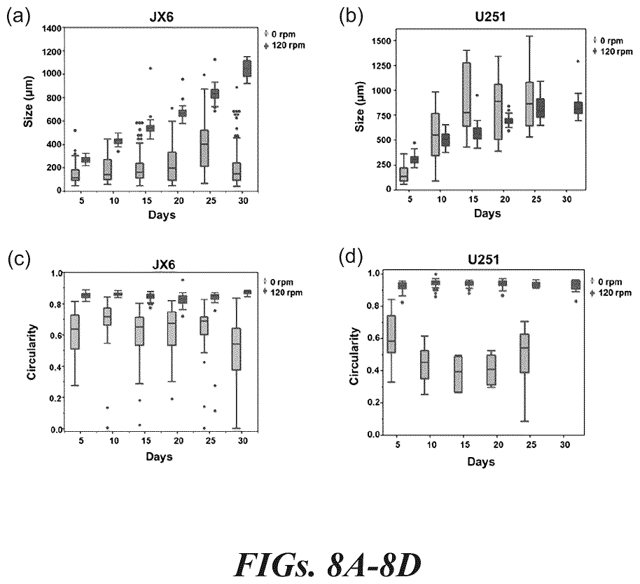

Example #2—Stable Production of PDX GBOs over Millimeter Scale: It was previously shown that millimeter scale brain organoid model can drive stem cell differentiation, subsequently organizing into differentiated cell layers (Lancaster et al., 2013). Thus, GBOs of this scale may exhibit organized TME with high heterogeneity induced by GSC differentiation. To achieve millimeter-scale GBOs, U251 and PDX cell line JX6 were inoculated as single cells and continuously cultured in the bioreactor without dissociation over 30 days (FIGS. 7A-7C, FIGS. 8A-8D). Growth of the biorcactor GBOs (bGBOs, 120 rpm) were compared with static tissue culture flask cultured GBOs (tGBOs, 0 rpm) in terms of their size and circularity. Growth of U251 bGBOs slowed down after 3 weeks, whereas JX6 bGBOs became larger than 1 mm in diameter in 5 weeks (FIGS. 8A-8B). Most U251 and JX6 tGBOs were generated by random aggregations (FIG. 7A). Although the cells kept proliferating and increasing in number, the overall viability of both U251 and JX6 bGBOs gradually dropped over the culture period (FIG. 7B). It is most likely due to stresses from the hypoxic core developed inside the bGBOs. Variations in the bGBOs' size and circularity were significantly smaller than those of tGBOs for both cell lines (FIGS. 8C-8D). The high circularity indicates the bGBO production by cell proliferation, not by random aggregations. High circularity and the growth of the bGBO were not dependent on cell lines but the bioreactor culture system (FIG. 7C). Together, the large bGBOs were stably produced within 5 weeks by clonal proliferation. JX6 showed constantly higher proliferation compared to U251. Stable production of bGBOs was dependent on the bioreactor culture system regardless of the cell line.

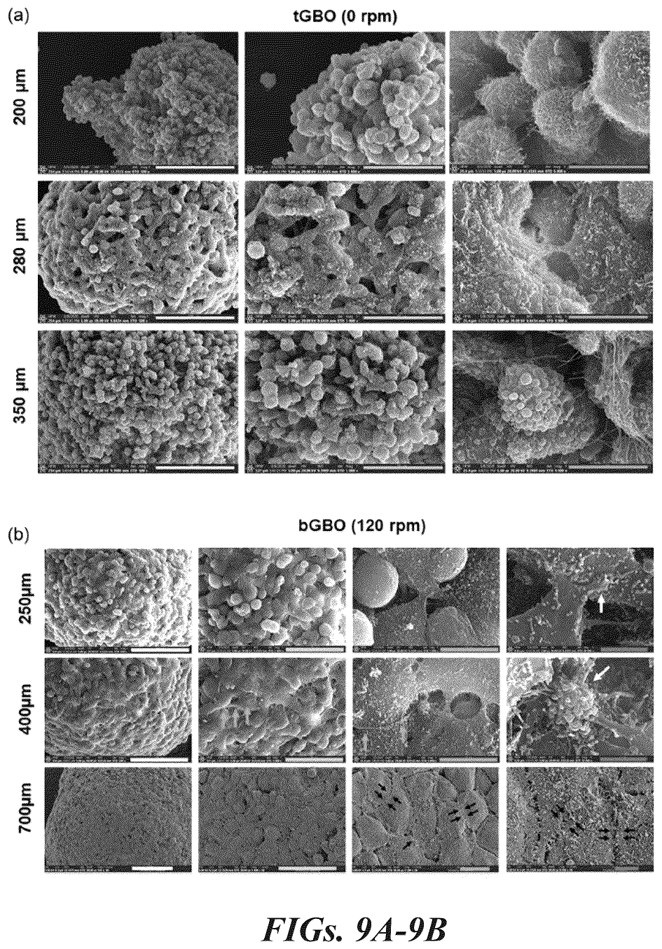

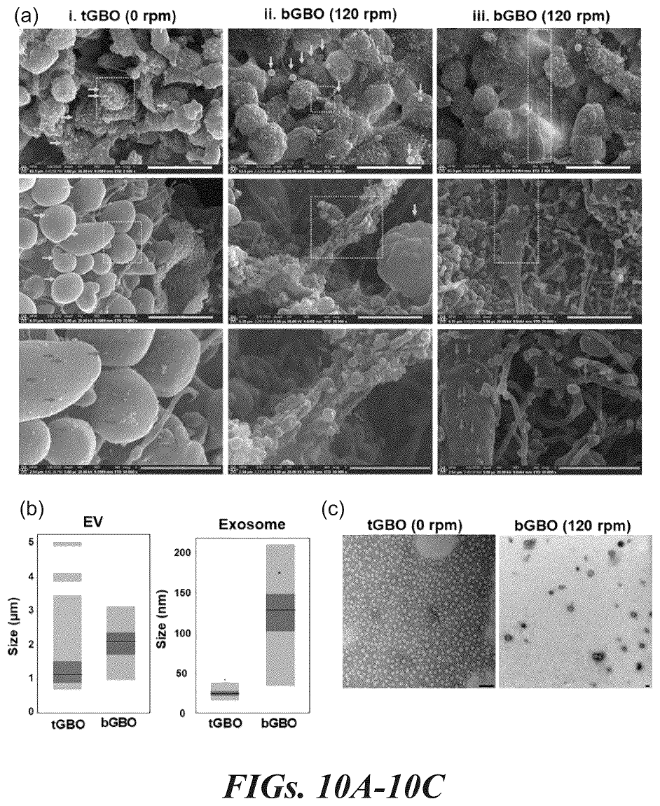

Example #3—Structural Integrity of Bioreactor-Produced GBOs by Cell-to-cell Interaction: To further investigate the GBO surface morphology in response to the shear stress of the biroeactors, tGBOs and bGBOs were imaged with SEM and compared (FIGS. 9A-9B, FIGS. 10A-10C). The bGBOs showed smoother surfaces and adherent cell morphology compared to tGBOs (FIGS. 9A-9B). The high cell-to-cell interactions were observed even when the size of the bGBOs was smaller than 300 μm (FIG. 9B). Cell-to-cell interactions were further strengthened by building bridges of lamellipodia (FIG. 9B, white arrows). Interestingly, cells on the bGBOs showed axon-like long protrusions of over 100 μm (FIG. 9B, cyan arrows). As the bGBOs grew, cell-to-cell interactions stitched over empty spaces, indicating tight junction (TJ)-mediated cell-to-cell interactions that resulted in smoother and flatter surfaces of the bGBOs (FIG. 9B, black arrows). This high and strong cell-to-cell interactions allowed the bGBOs to have high cell density, which mimic the dense cell population of in vivo GBM features.

Despite continuous culture of tGBOs without dissociation, the average size of tGBOs was limited to less than 500 μm. tGBOs greater than 1 mm in diameter were easily dissociated even with gentle media addition during feedings. This may be the result of the weak cell-to-cell interactions in the tGBOs that contributed to their structural fragility. To investigate the integrity of GBOs, morphology of the bGBOs and the tGBOs was analyzed by SEM (FIGS. 9A-9B). The tGBOs had rough surfaces, and cavities were present (FIG. 9A). Morphologically, cells in the tGBOs remained round, retained distinct individual cell shapes with vastly radiating microvilli and filopodia (FIG. 9A).

Together, the bGBOs exhibited structural integrity via strong physical cell-to-cell interactions. Constant agitation induced adherent cell morphology on the bGBO and axon-like long protrusions. Cell-to-cell interactions results in smoother surface of the bGBO and developed into TJs.

Example #4—Secretion of Extracellular Vesicles (EVs) and Exosomes: Extracellular vesicles (EVs) were previously found to be secreted by tumor cells as a means to modulate their TME and mediate cell-to-cell communication (Nakano et al., 2015). EVs can be heterogeneous both in size and in the type of cargos depending on what they carry. It has been suggested that GSCs employ EVs for intercellular communication. Since stem cell-enriching conditions were utilized, it was hypothesized that EVs would play an important role in developing GBOs (FIGS. 9A-9B, FIGS. 10A-10C).

Using SEM, clear formation of EVs was observed in both tGBOs and bGBOs (FIGS. 9A-9B). EVs secreted from the bGBOs were fully budded out and larger than the EVs from the tGBOs (FIG. 10A, yellow arrows). On the other hand, the tGBOs showed membrane ruffling and budding EVs on their surfaces (FIG. 10A, yellow arrows). Both GBO models secreted similar size of EVs (FIG. 10B). Many exosomes, which are smaller submicron subset of EVs, were also found on the surface of both GBO models, indicating that cell-to-cell communications were also mediated by them (FIG. 10A, pink arrows). The exosomes from bGBOs had more variety of sizes and were larger than those from the tGBOs (FIG. 10A, red arrows). Interestingly, EVs were found where physical cell-to-cell connections were formed with neighboring cells in bGBO (FIG. 10A (ii-iii), yellow box, red and pink arrows). These various sizes of exosomes secreted from the bGBOs were further verified by TEM analysis (FIGS. 10B-10C). The size distribution of the bGBO EVs was much greater than the tGBO EVs. Also, the average size of the bGBO EVs (118±34 nm) were significantly larger than the tGBO EVs (25±4 nm). Therefore, while both tGBOs and bGBOs secreted EVs, EVs from bGBOs promoted a more noteworthy formation of physical cell-to-cell interactions.

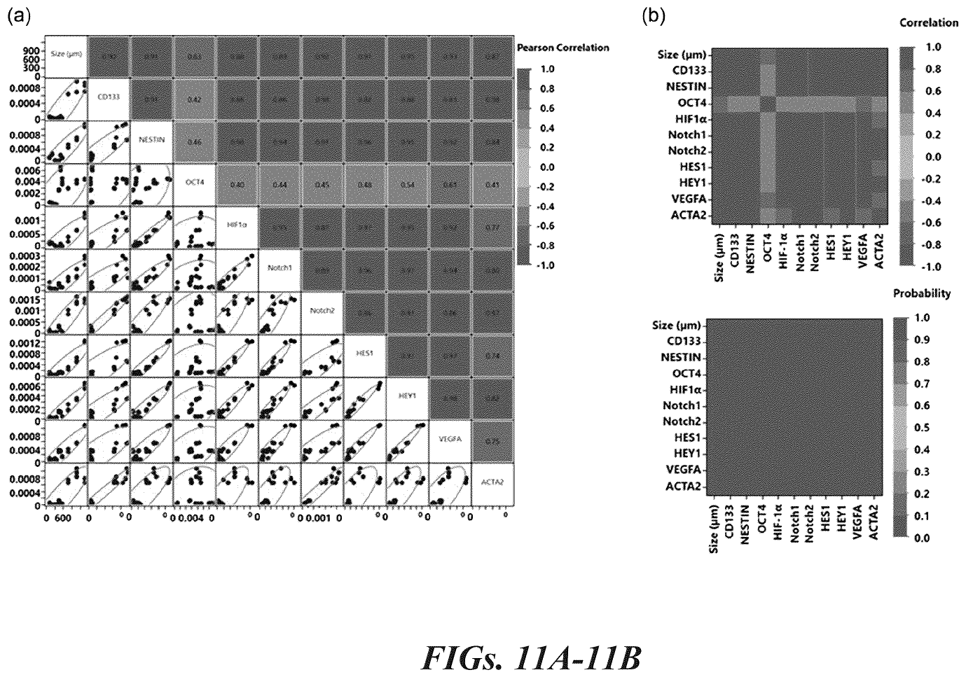

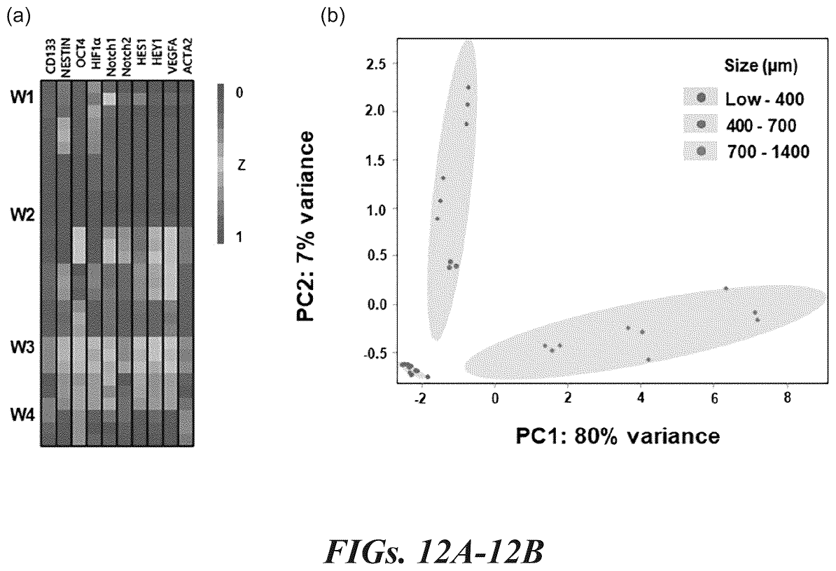

Example #5—Upregulation of Transcriptional GBM Phenotypes in Size-Dependent Manner: Next, the transcriptional phenotypes of the bGBOs were analyzed as a function of size to verify whether the bGBOs maintained their stemness and heterogeneous molecular profiles as they grow (FIGS. 11A-11B, FIGS. 12A-12B, FIG. 13, FIG. 14). Gene analysis showed strong positive correlation between the size of GBOs and gene expression, as well as a strong correlation among the various genes (FIGS. 11A-11B). This strong positive correlation indicates that the larger bGBOs exhibited higher stemness and heterogeneity. The gene expression sorted by duration of culture shows the strong positive correlation between the size and the gene expression was only evident by weeks 3 and 4 (FIG. 12A, W3 and W4). Further gene analysis revealed that this expression pattern was grouped by three different ranges of the bGBO sizes: single cells-400 μm diameter, 400 μm-700 μm diameter, and greater than 700 μm diameter (FIG. 12B).

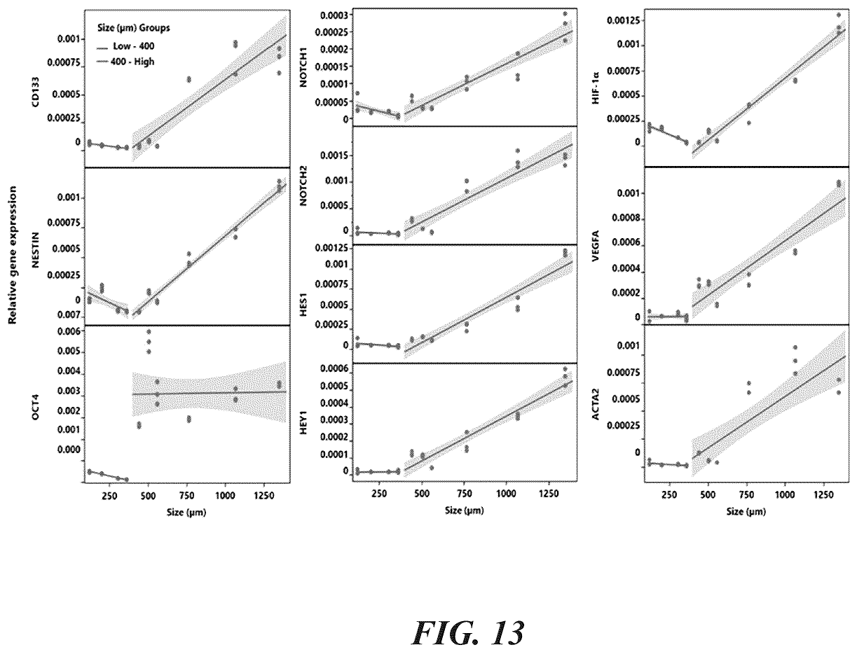

The correlation between the gene expression and the GBO size was further investigated (FIG. 12A). Strong positive correlation between the size and the gene expression started to emerge when the size of the bGBOs was greater than 400 μm in diameter (FIG. 13). Particularly, upregulation of HIF-1α suggests a new factor governing molecular profiles of the bGBOs. Recent study showed that in vitro tumor spheroids greater than 200 μm contain a hypoxic core (Barisam et al., 2018; Daster et al., 2017). More importantly, in vivo histopathological results demonstrated that the median distance between hypoxic area to the closest blood vessels is 130 μm (range, 80-200 μm) (Beasley et al., 2001). Thus, bGBOs larger than 400 μm in diameter were expected to start developing a hypoxic core, and the data corroborates with these prior studies.

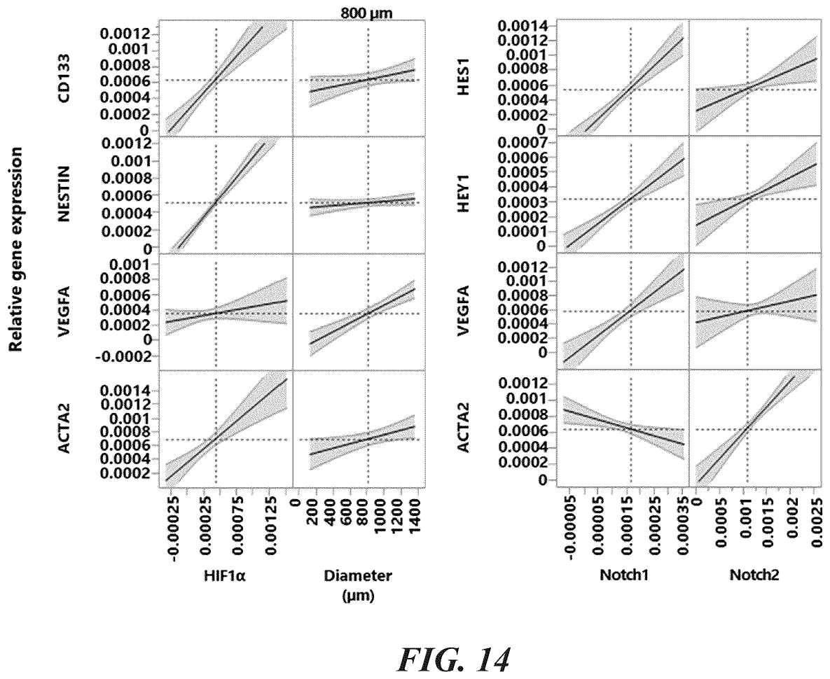

To better understand the interdependent correlation among the tested genes, statistical prediction model analysis was performed (FIG. 14). Upregulation of CD133 and NESTIN was highly associated with HIF-1α expression rather than the GBO size. Although the correlation between VEGFA and HIF-1α became positive when GBOs grew larger than 400 μm in diameter, VEGFA showed stronger correlation with NOTCH1 than with HIF-1α in the bGBOs. This indicates that the transcription of VEGFA was dominantly regulated by NOTCH signaling. HES1 and HEY1, downstream of NOTCH signaling, were more associated with NOTCH1 than NOTCH2. Interestingly, the expression of a pericyte marker ACTA2 was associated with NOTCH2, which indicates that NOTCH2 signaling may drive the GBM transdifferentiation into pericytes.

Collectively, these global gene expression analyses demonstrated that a strong correlation exists between the size of the bGBOs and their molecular profiles. Activation of stemness gene expression started when the size of the bGBOs was greater than 400 μm in diameter, mimicking the distance of diffusion limitation previously reported in vivo. HIF-1α was a key regulator governing GSC molecular profiles. The upregulation of CD133 along with HIF-1α suggests that the GSCs are results of HIF-1α activation. Finally, the NOTCH upregulation was associated with angiogenesis and transdifferentiation of GBM.

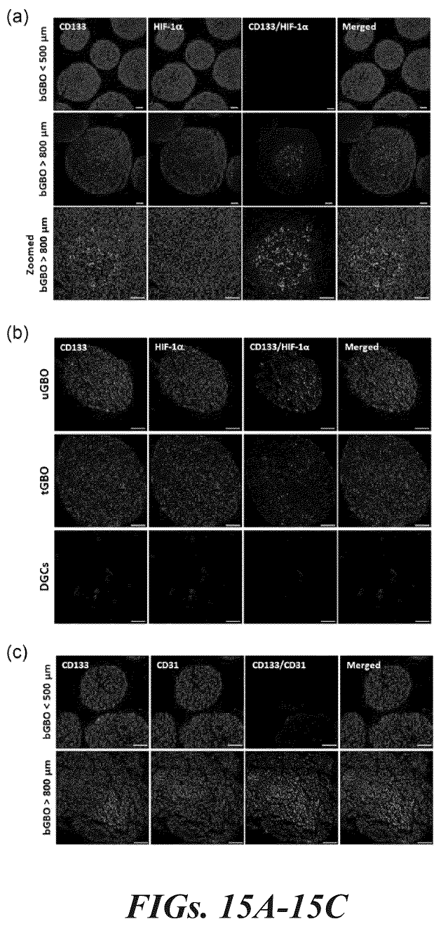

Example #6—Self-Established GSC Niches: Given the global gene expression analysis (FIGS. 11A-11B, FIGS. 12A-12B, FIG. 13, FIG. 14), it was hypothesized that the bGBOs with greater than 400 μm diameters start reprogramming themselves to build a heterogeneous TME via HIF-1α and NOTCH. To examine this hypothesis, different sizes of bGBOs were compared with tGBOs, with GBOs produced by forced cell aggregations in U-bottom well (uGBOs) and with serum-induced differentiated glioblastoma cells (DGC). Small bGBOs (smaller than 500 μm diameter) have no sign of a hypoxic core confirmed by HIF-1α or CD133-positive GSCs (FIG. 15A). On the other hand, large bGBOs (greater than 800 μm diameter) displayed the development of a hypoxic core shown by HIF-1α-positive region where the cells expressed CD133. The area where CD133 expression overlapped with HIF-1α recapitulated in vivo histopathological features of the hypoxic niche. Large uGBOs and tGBOs also had CD133-positive cells, but their CD133-positive cells were not spatially organized and not overlapped with the HIF-1α-positive region (FIG. 15B). DGCs did not express CD133 and HIF-1α proteins.

Hypoxia induces transdifferentiation of GSCs into endothelial cells for tumor vascularization (Ricci-Vitiani et al., 2011; Rocha et al., 2018). Many studies showed that CD133-positive cells exhibit endothelial cell phenotypes and co-express CD31 in vivo (Christensen et al., 2011; He et al., 2012; Hilbe et al., 2004). This in vivo GBM feature was confirmed in the bGBOs. Cells expressing CD31 resided near the CD133-positive cells, and CD133/CD31 double positive cells were observed (FIG. 15C). The presence of CD31-positive cells indicates that the bGBOs were capable of tumor vascularization and transdifferentiation into endothelial cells.

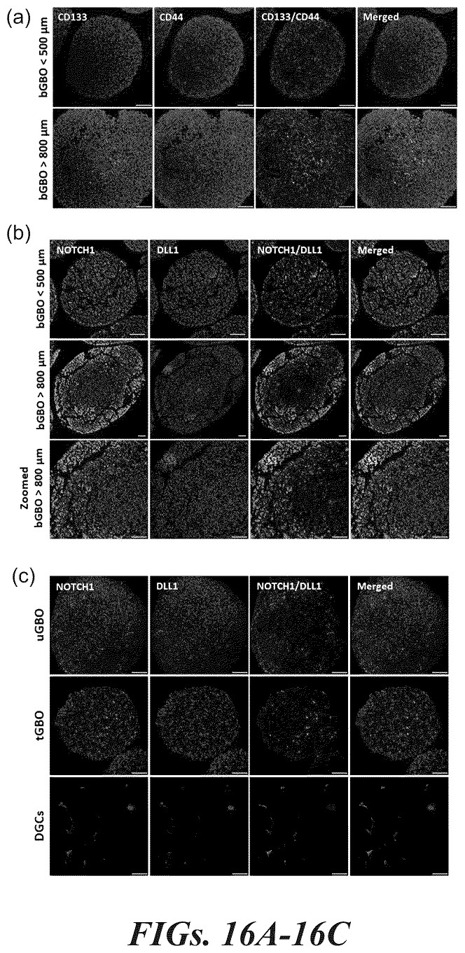

The expression of CD44, a tentative GSC marker, and its spatial variation in the bGBOs were also examined (FIG. 16A). It was reported that CD44 collaborates with CD133 in GBM and is highly correlated with tumorigenesis (Brown et al., 2017). CD44 was expressed throughout the bGBOs, but its expression did not overlap with the CD133- and HIF-1α-positive expression area. Thus, CD44 expression is likely not associated with hypoxia. Distinctive CD44 and CD133 singly positive cells indicate a heterogeneous GSC population emerged.

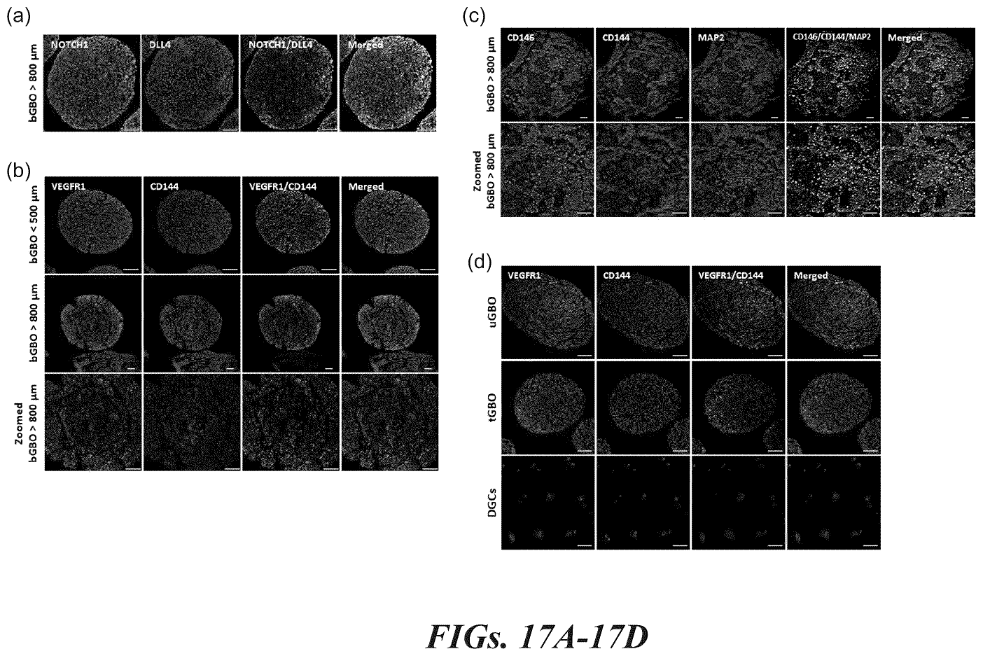

Another GSC marker of NOTCH1 and its ligand proteins DLL1 and DLL4 were also analyzed to further investigation of gene analysis. Large bGBOs showed doughnut-shaped gradient expression of NOTCH1 (FIG. 16B). NOTCH1 expression diminished near the center of the organoid where hypoxic niche was observed (FIG. 15A). This phenomenon agrees with many studies which showed that NOTCH is preferentially expressed near the perivascular niche where oxygen and nutrients are sufficiently provided (Bayin et al., 2017; Charles et al., 2010; Zhu et al., 2011). Likewise, the expression of NOTCH was high near the surface of the bGBOs where mass transfer was not limited. This is in contrast to the center of the GBOs where there were significant diffusion limitations. Thus, the organoids mimicked the perivascular niche found in vivo. NOTCH1 ligands DLL1 and DLL4 were spatially and highly expressed in the NOTCH-positive region, suggesting the activation of NOTCH signaling (FIG. 16B, FIG. 17A). The tGBOs and DGCs showed poor expression of both markers (FIG. 16C).

It has been reported that NOTCH-DLL signaling plays an important role in angiogenesis, and inhibition of the signaling sensitizes GBM to anti-VEGF therapy (Benedito et al., 2009; Hellstrom et al., 2007; Li et al., 2011). Whether NOTCH-DLL signaling is spatially correlated with VEGF expression, VEGF receptor-1 (VEGFR1) and its spatial organization were examined (FIG. 17B). VEGFR1 expression displayed a similar pattern of NOTCH1 in the large bGBOs. Interestingly, there was a clear border between vasculogenic differentiation marker CD144-positive cells and VEGFR1-positive cells. It was reported that the signaling of CD144 to VEGFR 1 mediates formation of vasculogenic mimicry (VM) (Frank et al., 2011). The formation of VM in the GBOs was verified by the co-expression of CD144 with CD146 and MAP2 (FIG. 17C). Thus, CD144-mediated VM formation in the large bGBOs can be a potential in vitro model for tumor vascularization. In contrast, the uGBOs and the tGBOs did not show clear spatial expression of VEGFR and CD144 (FIG. 17D).

Taken together, the bGBOs mimicked the in vivo spatial organization of GBM, namely the hypoxic niche and the perivascular niche. VEGFR 1 expression was highly associated with NOTCH1 expression pattern, which was consistent with the gene expression analysis. The transdifferentiation of GBM into endothelial cell phenotypes and formation of VM supported by pericyte and astrocyte was also confirmed. Size-dependent spatial expression and transdifferentiation can be the result of GSC plasticity, which recapitulates heterogeneous in vivo GBM TME features.

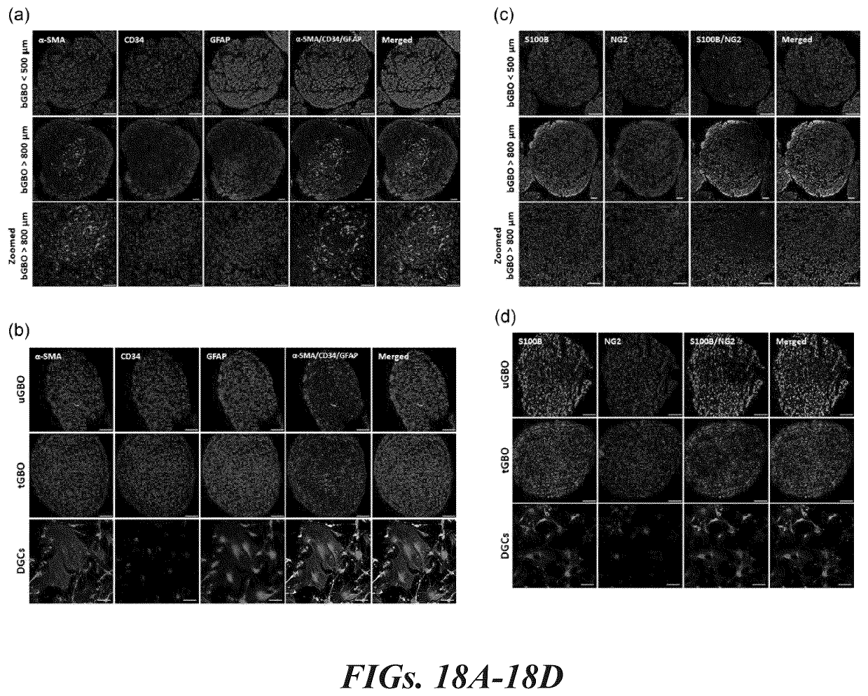

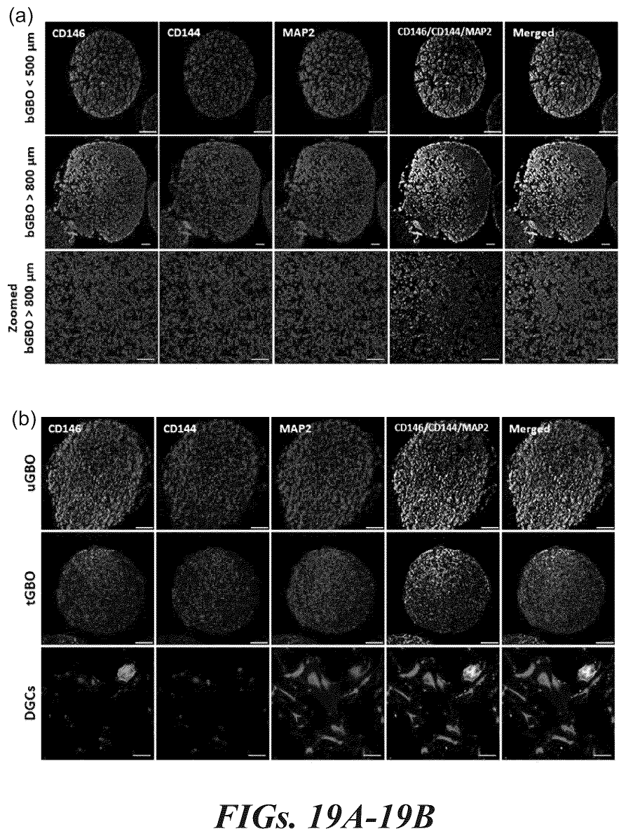

Example #7—Establishment of GBM TME Driven by Transdifferentiation of GBM into Endothelial Cells, Pericytes, and Astrocytes: Global gene expression analysis of the GBOs above revealed that the transdifferentiation of GBM into pericytes was regulated by increased NOTCH expression (FIGS. 11A-11B, FIGS. 12A-12B, FIG. 13, FIG. 14) and that cells transdifferentiated into endothelial cells (FIGS. 15A-15C, FIGS. 16A-16C, FIGS. 17A-17D). The transdifferentiated cells may play a role in establishing heterogenous GBM TME, thus, the presence of transdifferentiated GBM and their spatial distribution were further analyzed. To investigate transdifferentiation, IHC analysis of GBOs was performed with endothelial cell markers (CD34 and CD144), pericyte markers (α-SMA [ACTA2], NG2, and CD146), and astrocyte markers (S100B, MAP2, and GFAP). In the large bGBOs (greater than 800 μm diameter), α-SMA expression showed the most distinctive spatial variation, encircling the core region where hypoxia was present (FIG. 18A). In contrast, α-SMA was not expressed in the small bGBOs (smaller than 500 μm diameter), which agrees with the prior gene analysis. The cells expressing α-SMA had an elongated morphology similar to DGCs' morphology (FIG. 18B). The uGBOs had only a few cells expressing α-SMA and no patterns of spatial organization, whereas the tGBOs showed poor expression of α-SMA (FIG. 18B). Other pericyte markers (i.e., NG2 and CD146) also did not display spatial expression in the small bGBOs (FIG. 18C, FIG. 19B). NG2 expression was poor in the uGBOs and the tGBOs compared to bGBOs. Particularly, the tGBOs did not show NG2 expression.

The large bGBOs contained strongly positive for CD34 and CD144 cells near the core region (FIG. 18B, FIG. 19A). The expression of CD34 and CD144 was not induced by serum (FIG. 18B, FIG. 19B). Interestingly, CD146/CD144 double positive cells were observed in the large bGBOs (FIG. 19A). This supports other studies of the receptor profiles of endothelial progenitors that share pericyte and endothelial cell markers (Desai et al., 2009; Hristov et al., 2003; Park et al., 2020; Timmermans et al., 2009). Thus, both CD146- and CD144-positive cells may represent endothelial progenitors.

Astrocyte markers GFAP, S100B and MAP2 also showed spatially gradient expression. As GBM is a grade IV astrocytoma, the expression of these markers is natural. However, the gradient expression of these markers with the size increase of the bGBOs indicates the process of spatial organization.

Collectively, the millimeter scale bGBOs featured spatially organized and transdifferentiated GBM cells. Protein expression and spatial variation became clear in the larger size GBOs. Although endothelial cell markers expression was not as clear as some of the other markers, the expression levels of endothelial cell markers were significantly higher than those of tGBOs and DGCs.

Example #8—Hierarchically and Spatially Organized GSC Niche with Tumor Necrotic Region: A necrotic area is a common histopathological GBM feature and governs signaling pathways in the GBM TME (Ahn et al., 2016; Liu et al., 2017; Rong et al., 2006). Necrotic regions commonly accompanied with a hypoxic environment create inflammatory microenvironments. For example, TNF-α induces TGF-β, which subsequently induces α-SMA expression (Desmoulicre et al., 1993; Hu et al., 2003; Warshamana et al., 2001; Yoshimatsu et al., 2020). To model these necrotic environmental features, bGBOs larger than 1.5 mm in diameter were characterized.

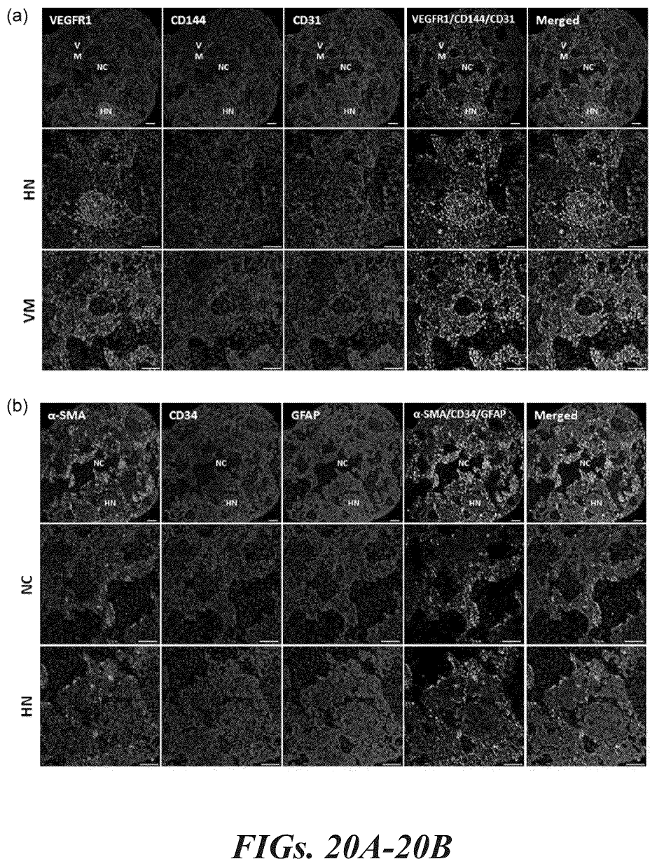

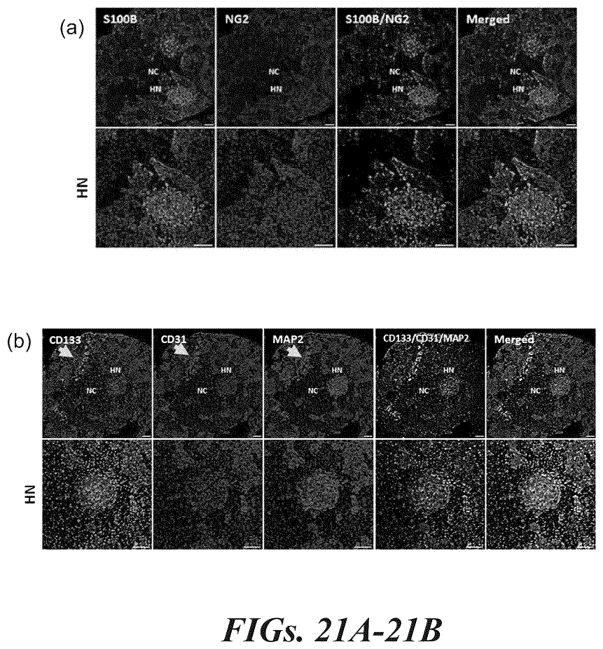

These bGBOs greater than 1.5 mm diameter exhibited a large necrotic core near the center where lots of DNA fragments were observed (FIGS. 20A-20B, FIGS. 21A-21B, FIG. 22A-22B, NC). In addition to the necrotic core (NC), formation of VM was confirmed by shape and high expression of α-SMA, CD144, and CD31 on the border of the labeled region (FIG. 20A, VM). Spheroid shape of distinctive GBM TME was localized near the NC (FIGS. 20A-20B, FIGS. 21A-21B, FIG. 22A-22B HN; hypoxic niche). This area exhibited a high expression of CD133, VEGFR1, GFAP, and MAP2. The expression of CD133 accompanied with the high and distinctive spatial expression of VEGFR1 suggested a potential GSC niche possessing angiogenesis feature. Some CD31-, CD34-, and CD144-positive cells were found in the HN area; however, these markers were more expressed around the circled area, which may play roles in supporting CD133-positive cells (FIG. 21B). The HN area suggests a potential GSC niche accompanied with endothelial cells supporting GSC niche.

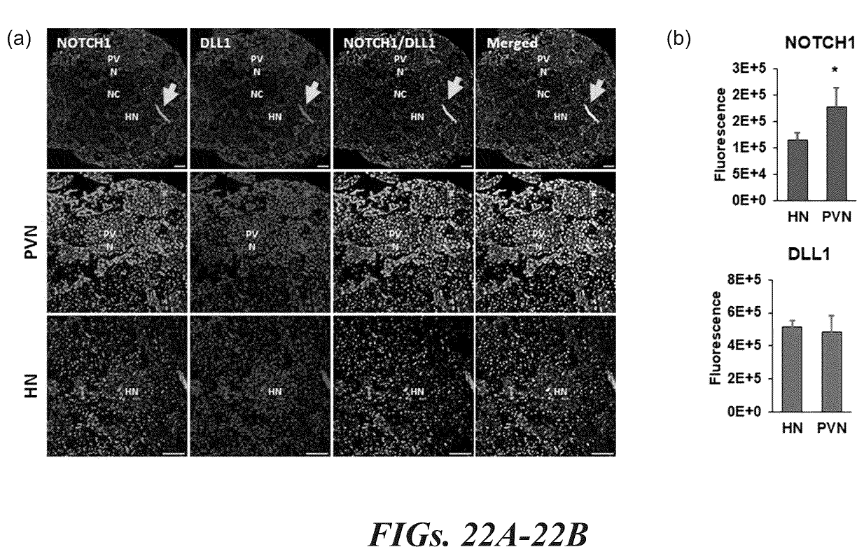

NOTCH1 expression was poor in the hypoxic niche, but another potential perivascular niche model was found where NOTCH1 expression was high (FIG. 22A, PVN). In this area, cells expressing NOTCH1 also formed a spheroid shaped colony like the potential hypoxic niche. This perivascular niche is within 200 μm from the surface of the bGBOs where there is enough oxygen and nutrients. This gradient expression of NOTCH1 was confirmed again, which agrees with FIG. 16B, indicating reproducible and spatially organized heterogenous GBO models.

α-SMA displayed the most distinctive spatial expression (FIG. 20A). Expression of α-SMA was high along the necrotic core. This spatial expression of α-SMA can be the result of cytokines released in an inflammatory environment due to the necrotic core. Interestingly, the potential GSC hypoxic niche was devoid of α-SMA (FIG. 20A). Thus, it is most likely that cells expressing α-SMA were fully differentiated. NG2 expression also diminished in the HN region. However, this area was strongly positive for S100B and MAP2. The distinctive spatial expression of astrocytes and pericytes indicates established GBM TME supported by fully differentiated cells.

Together, the necrotic GBM TME shows multiple hierarchically and spatially organized GSC niches. These GSC niches were identified by distinct areas of CD133- and NOTCH1-positive cells. Notably, the niches were void of pericyte and endothelial cell markers. The hierarchical organization was highlighted by pericytes near the necrotic core and endothelial cells surrounding the CD133-positive GSC niche area.

Example #9—Self-Assembled GBM TME Driven by High Cell-to-Cell Interactions and Activation of POU3f2 and HIF-1α: bGBOs exhibited strong and physical cell-to-cell interactions supported by TJs (FIGS. 9A-9B, FIGS. 10A-10C). Therefore, it was hypothesized that the regulation of cell-to-cell interactions mediates spatial organization of GBM TME. TJ plays important roles in intercellular interactions, invasion, and blood brain barrier (BBB) permeability (Bhat et al., 2019). TJ not only mediates differentiation and proliferation but also plays a role in maintaining the integrity of organoid models and stemness (Matter et al., 2005; Xing et al., 2020; Xu et al., 2012).

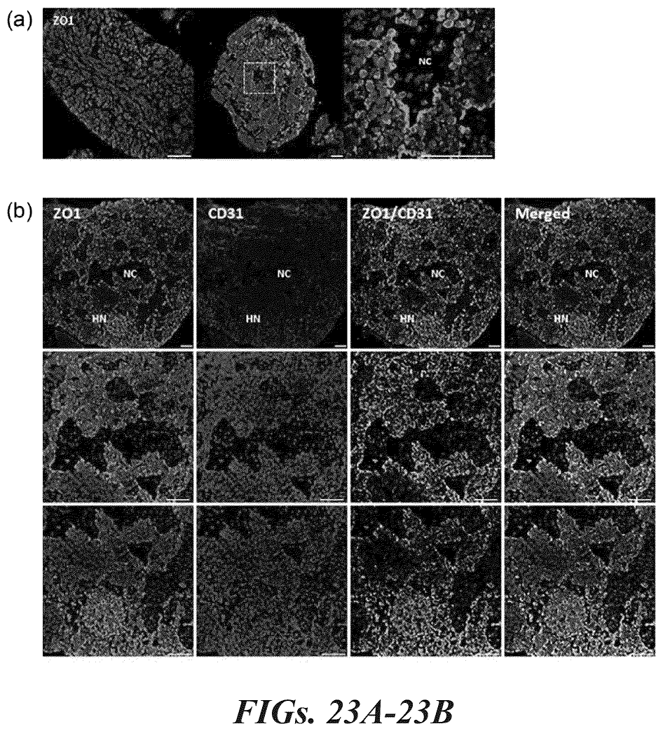

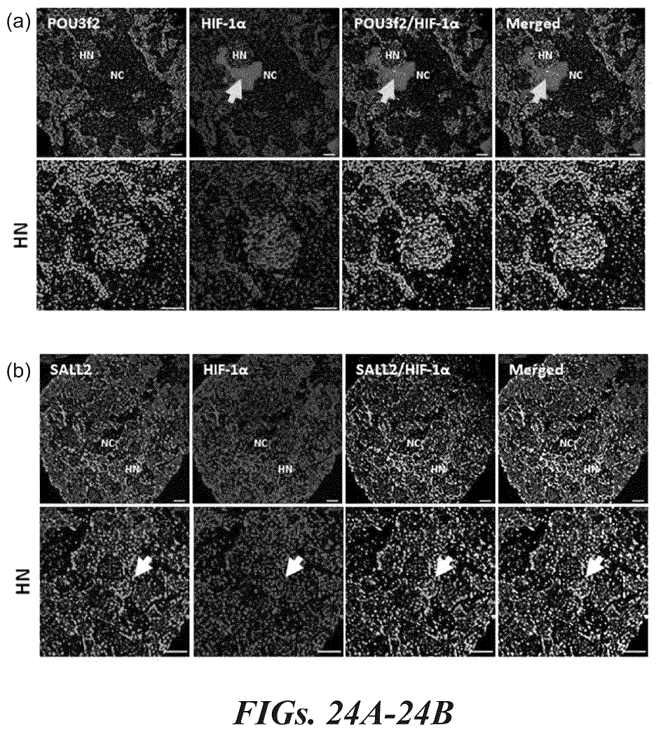

To examine the hypothesis, different sizes of the bGBOs were stained by ZO-1, a TJ protein (FIG. 23A). Cells having an elongated morphology were actively migrating in the center region of the bGBOs when they were greater than 500 μm in diameter, which starts developing a hypoxic core as shown in FIGS. 15A-15C, FIGS. 16A-16C, and FIGS. 17A-17D. It was reported that hypoxia decreases expression of TJ and allows cells to have high mobility (Luo et al., 2018). Thus, the highly elongated cells displaying lower ZO-1 expression can be in response to the hypoxic TME. In contrast, in larger bGBOs, ZO-1 was highly expressed on the boundary of the small necrotic regions, which allows for clear internal structure of the bGBOs (FIG. 23B). The formation of TJ along the necrotic areas may protect the cells from acidic and hypoxic environmental factors including inflammatory cytokines which cause apoptosis. Interestingly, the potential hypoxic niche shown in FIGS. 20A-20B, FIGS. 21A-21B, and FIG. 22A-22B highly expressed ZO-1 but not for CD31 (FIG. 24A). It was reported that ZO-1 expression decreased during metastasis, but cancer cells recover ZO-1 expression in metastasized site (Kaihara et al., 2003). Also, ZO-1 knockout stem cells failed to form spheroids in vitro (Xing et al., 2020). Therefore, the spheroid shape of the potential hypoxic niche and the high expression of ZO-1 provide evidence of a newly and hierarchically established GSC niche in the bGBOs.

GSCs in their niche can be differentiated or dedifferentiated by activation of transcription factors. POU3f2, SALL2, and HIF-1α have been reported to induce dedifferentiation of GSCs (Lee et al., 2016; Suva et al., 2014). To better understand the underlying mechanisms of CD133-positive cells shown in FIGS. 20A-20B, FIGS. 21A-21B, and FIG. 22A-22B, expression of POU3f2, SALL2, and HIF-1α was examined. In the hypoxic niche, cells highly expressed the three transcription factors, suggesting that CD133 expression was maintained by POU3f2, SALL2, and HIF-1a.

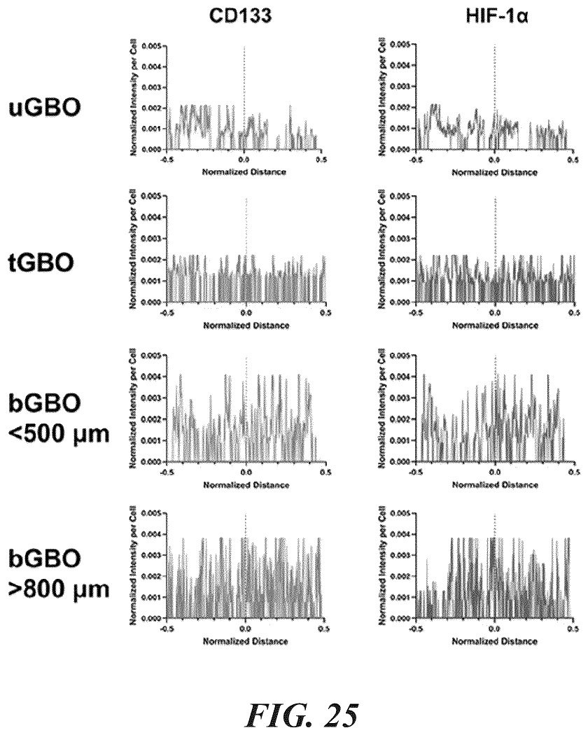

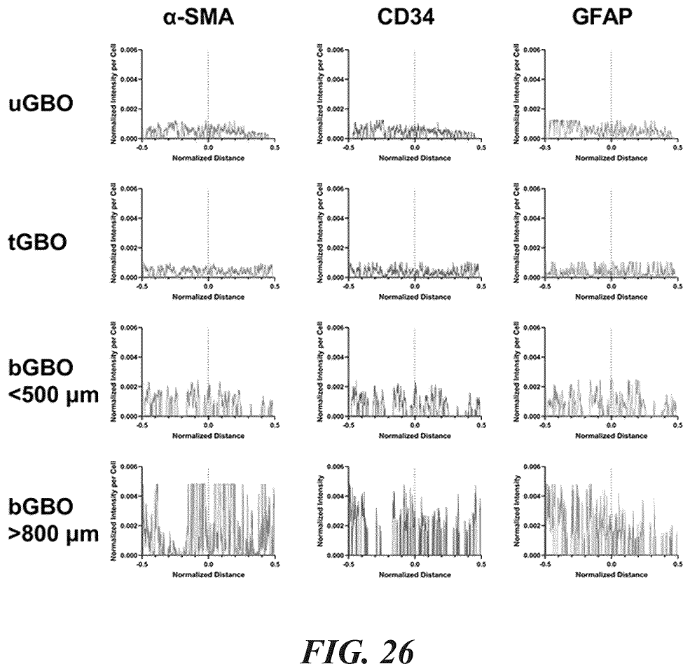

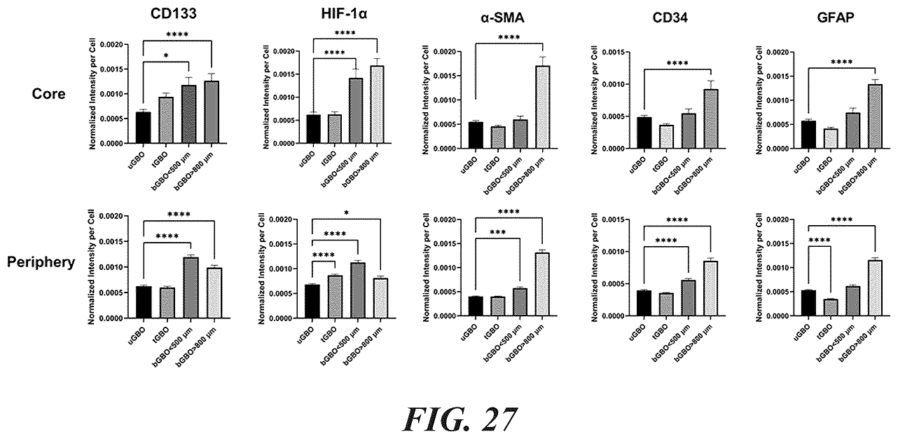

Collectively, it is proposed that the establishment of the GSC niche in the bGBOs was driven by cell-to-cell interactions and activation of transcription factors involved in GSC dedifferentiation (FIG. 25, FIG. 26, FIG. 27). Spatial organization was initiated by downregulating ZO-1 near the center. Near the necrotic regions, the expression of ZO-1 was high and may protect cells from the inflammatory factors and acidic TME. In the bGBO model, the hypoxic niche was spatially and hierarchically organized by activation of POU3f2, SALL2, and HIF-1a.

Example #10—Scalable Biomanufacturing of Tumor Organoids Protocols: Glioblastoma multiforme (GBM) is the most common and aggressive primary brain tumor in adults with a prognosis of 12-15 months. The aggressiveness of GBM is likely attributed to its heterogeneity of the tumor microenvironment (TME). Current in vitro models of GBM offer limited translation to in vivo tumors because of the heterogeneous TME, leading to limited progress in drug development and treatment. Organoids are millimeter-scale, 3D in vitro modeling systems that recapitulate stem cell differentiation into highly organized, heterogeneous in vivo organ-like systems. This study used defined engineering parameters on single GSCs to grow and develop into GBM organoids (GBOs) with a diameter up to 4.0 mm in three months or less, without the use of media-induced differentiation or extracellular matrix (ECM) mimicking scaffolds. The study found that the GBOs self-induced differentiation and organized cells into some key components of the GBM TME (i.e. hypoxic niche, angiogenesis, and necrotic core). Interestingly, the GBOs also self-secreted and organized its own key ECM components (i.e. collagen IV, elastin, laminin, and hyaluronic acid). These studies aim to understand what induces GSC differentiation and ECM redesign to form the heterogeneous TME and how this dynamic TME leads to varying drug response.

Neurobasal Media Formulation (optimized for neural progenitor cell growth): The following was added to one 500 mL Neurobasal media bottle:

TABLE 3
Neurobasal Media Formulation

| Product Name (link) | | Volume (to add to 500 |
|---|---|---|
| Manufacturer, Cat# | Purpose | mL media bottle) |
| Neurobasal ™-A Medium | Base media formulation | 500 mL |
| Gibco ™, 10888022 | | |
| GlutaMAX ™ Supplement, | Amino acid supplement primarily for auxiliary | 2.5 mL |
| 100X | energy source. Used instead of 1-glutamine due | (working |
| Gibco ™, 35050061 | to stability. | concentration = 0.5x) |
| B-27 ™ Supplement (50X), | Serum-free supplement for neural progenitor | 5 mL |
| minus vitamin A | and stem cells without the need for an | (working |
| Gibco ™, 12587010 | astrocyte feeder layer | concentration = 0.5x) |
| Penicillin-Streptomycin | Antibiotic mix of Penicillin (10,000 IU/mL) | 5 mL |
| Solution, 100x | and Streptomycin (10,000 μg/ml). Abbreviated | (working |
| Corning ™, 30-002-CI | as pen/strep. | concentration = 1x) |
| OR 1 mL Gentamicin | | |
| Sulfate (50 mg/mL) | | |
| Corning ™, 30-005-CR | | |
| 50 mL Amphotericin B, | Antifungal supplement. | 5 mL |
| Liquid, 250 μg/mL | Abbreviated as Amph. B. | (working |
| Corning ™, 30-003-CF | | concentration = 2.5 |
| OR 500 μL Fungizone | | μg/mL) |
| (1000X) | | |
| Corning ™, 30-005-CR | | |
| Heparin sodium salt* | Aids in binding VEGF and FGF due to | 1 mL of stock aliquot* |
| CAS Number: 9041-08-1 | negative charge motifs on its side chain. | (working |
| Sigma-Aldrich ™, | Abbreviated as Hep. | concentration = 8 |
| H3149-10KU | | μg/mL) |
| Recombinant Human EGF* | Epidermal growth factor (EGF) helps with | 1 mL of stock aliquot* |
| Shenandoah Biotechnology, | survival and proliferation of glioma stem cells. | (working |
| 100-26-1 mg | | concentration = 20 |
| | | ng/mL) |
| Human FGF-basic 154 aa | Basic fibroblast growth factor (bFGF) helps | (working |
| (FGF2)* Shenandoah | with survival and proliferation of stem cells. | concentration = 20 |
| Biotechnology, 100-146-1 mg | | ng/ml) |

*See aliquot protocol below.

### Determining Spheroid Critical Threshold Size (CTS):

First, the desired suspension cell line was passed in serum-free media. It is essential that single cells are healthy prior to beginning organoid development. Here, healthy suspension cells are defined as cells that grow in aggregates, that are easily dissociated, and that exhibit high viability. Easy dissociation includes dissociation of aggregate with simple shear stress, often through pipette trituration, and enzymatic dissociation using Accutase or trypsin for 3-5 minutes. High viability is greater than or equal to 90% viability.

Next, the cells were inoculated in a U-bottom shaped 96-well plate, V-bottom shaped 96-well plate, or microwell plate with a seeding density of 10⁵ live cells per 1 mL of media (104 live cells per 100 μL in a 96 well plate) to form spheroids. The U-bottom shaped 96-well plate was used for troubleshooting. The cells were then moved onto V-bottom shaped 96 well plates before moving into the microwell plate. For the scale-up process, the microwell plate was used to make spheroids.

Next, the size of the spheroids was measured on a light microscope. Images were taken to record the size and circularity of spheroids until CTS was determined. Next, samples of spheroids with a diameter of 100 μm to 1000 μm were taken, and relative gene expression for CD133 (cancer stem cell gene), HIF1α (hypoxia gene), NESTIN, OCT4, SOX2 (stem cell regulation genes), and other genes that regulate differentiation according to the specific cell line were studied. The relative gene expression was related to the size of the spheroids, and the size of the spheroids in which there was an observed upregulation of the genes listed above was determined.

If the spheroids showed upregulation with a diameter of 100-600 μm, and circularity of >80%, then the size in which upregulation began was determined to be the real critical threshold size (CTSᵣₑₐₗ). Critical threshold size (CTS) was defined as a range, with the minimum value being the CTSᵣₑₐₗ and the maximum value being CTSᵣₑₐₗ+200 μm.

Starting a Bioreactor from Spheroids: This protocol was used for 96-well plate derived spheroids to be inoculated in a 100 mL bioreactor with a total working volume of 60 mL and max shear stress at the wall range (τₘₐₓ) being 0.2-0.6 Pa (60-120 rpm). Following the same process and results from the above protocol for determining spheroid critical size, the desired suspension cell line was passaged in serum-free media. Single cell samples of 5×10⁶ live cells were taken after passaging to keep relative live cell counts consistent across all organoid samples.

According to results from the above protocol for determining spheroid critical size, the cells were inoculated in a U-bottom shaped 96-well plate, V-bottom shaped 96-well plate, or microwell plate with a seeding density of 10⁵ live cells per 1 mL of media (104 live cells per 100 μL in a 96 well plate) to form spheroids. Three more 96-well plates were seeded as control samples to keep live cell count consistent when samples were taken.

The spheroid plate was then incubated until time had passed for the spheroids to reach the critical threshold size (CTS) range. The size of the spheroids was checked under the microscope every day or every other day. It was verified that the spheroids had reached their CTS range by taking 20-30 images on the EVOS M5000 microscope, then measuring their diameter and circularity. The spheroid plate was then placed back in the incubator until ready to transfer.

An autoclaved/sterilized bioreactor was next placed in the sterilized biohood. The top of the bioreactor was gently taken off, and the bioreactor magnet and baffle were adjusted such that it was not touching the dimple at the bottom of the bioreactor. It was ensured that the magnet was balanced to prevent an uneven distribution of shear stress. Next, approximately 100 mL of sterile deionized water (DI·H₂O) was poured into the bioreactor. The lid of the bioreactor was replaced and closed tightly. The bioreactor was then placed in the incubator on the magnetic stir plate as described in the below protocol for removing/replacing the bioreactor to allow any small, solid particles to rinse out of the bioreactor. Rinsing lasted for at least 5 minutes. It was next ensured that the magnetic stir plate was set at the desired rpm for the desired shear stress. These steps were then repeated with a second bioreactor.

While the bioreactor was rinsing in DI·H₂O, the pre-imaged and analyzed spheroid plate was placed the biohood. Using a cut 100 μL pipette tip, 48 spheroids were then collected into one 15 mL centrifuge tube, and the other 48 spheroids were collected into a different 15 mL centrifuge tube. The 15 mL centrifuge tubes were each gently rocked back and forth to ensure no spheroids were stuck to the sides of the tubes. The spheroids were allowed to settle to the bottom of the tube naturally via gravity. Next, the supernatants were aspirated, leaving approximately 1 mL of supernatants to ensure no spheroids were lost in the transfer process. The spheroids were not centrifuged, so that there would be no effect on the viability and morphology of future organoids.

Next, the bioreactor rinsing with DI·H₂O was retrieved from the incubator and placed in the biohood. The bioreactor was gently opened from the top, and the magnetic stir bar, baffle, and the glass rod region of the bioreactor were cleaned with the vacuum aspirator. It was then ensured that the magnetic stir bar was evenly distributed across the baffle, then the top was tightly screwed back onto the bioreactor. The stir bar and baffle were confirmed to not be unbalanced/lopsided or touching the dimple at the bottom of the bioreactor. The filter cap was then removed, the DI·H₂O was poured out into a glass beaker, and the filter cap was tightly screwed on.

Any remaining liquids in the bioreactor were next aspirated out with the vacuum aspirator. Next, the bioreactor was refilled with 60 mL of fresh, warm (37° C.) Neurobasal (NBE) media. A cut 1 mL pipette tip was used to seed all 48 spheroids from the 15 mL centrifuge tube into the bioreactor. The extra 1 mL of media remaining in the centrifuge tube was then added to the bioreactor. The centrifuge tube was washed with 1 mL of media from the bioreactor to ensure all spheroids were seeded. Finally, the bioreactor was placed in the incubator as described in the below protocol for removing/replacing the bioreactor from the incubator.

Removing/Replacing Small Scale Bioreactor from Incubator Protocol:

First, the incubator doors were opened and the magnetic stir plate was turned off. The bioreactor of interest was removed or replaced from the plate and placed in the sterilized biohood. It was then ensured that the remaining bioreactors on the plate were still directly over the magnetic stir bar location on the plate (i.e., the dimple of the bioreactor was aligned with the X on the plate), and the magnetic stir plate was turned back on. The remaining bioreactors were checked to confirm that the stir bar was still rotating without a lag, splashing, or bubbles forming. Finally, the incubator door was closed gently so the bioreactors did not move around on the plate.

Bioreactor Maintenance and Imaging Protocol: This was done at least once per week at minimum. Organoids >1500 μm diameter needed media changed twice per week. First, the EVOS M5000 microscope was wiped down with ethanol (EtOH) and turned on to warm up while the organoids were collected for maintenance imaging. All organoids were then allowed to settle to the bottom of the bioreactor and gently swished around so they all aggregated towards the edge of the bioreactor. This made it easier to collect all the organoids while avoiding debris in the bioreactor.

Next, the bioreactor was opened from the top, and the magnetic stir bar, baffle, and the glass rod region of the bioreactor were cleaned with the vacuum aspirator. Next, in preparation for organoid collection, a petri dish was filled with 5-6 mL of fresh media. To collect the organoids, a cut 1 mL pipette tip was slowly hovered just above the organoids to pipette the organoids into the pipette tip. This was done very slowly to prevent the organoids from getting stuck to each other or to the pipette tip in this process. It was occasionally necessary to “pre-wet” the pipette tip to prevent organoids from attaching themselves to the pipette tip. The organoids were allowed to slowly escape the pipette tip by submerging it into the media collected in the petri dish. Larger organoids sometimes required gentle pipetting out and extra media to stay fully submerged, so some extra media volume was occasionally pipetted into the petri dish (though the petri dish was not filled with more than 7 mL of media). These steps were then repeated until all organoids were in the petri dish. Cellular debris or broken off organoids that were significantly (i.e., less than half) smaller were excluded.

The spent media was aspirated out using the vacuum aspirator. 50 mL of the spent media was removed from the bioreactor with a serological pipette and placed in a 50 mL centrifuge tube. The spent media was centrifuged for 5 minutes at 300 rcf (xg) and 4° C. Approximately 45 mL (up until freeze line in 50 mL tube) was removed from the centrifuged tube and placed into a new, fresh tube. The spent media was stored in 4° C. for up to 6 months or −20° C. for up to 12 months.

Next, sterile deionized water (DI·H₂O) was poured into the bioreactor up to the 100 mL mark in the bioreactor. The DI·H₂O was swished around in the bioreactor to collect some of the condensate on the sides of the bioreactor, then the top of the bioreactor was replaced and closed tightly. The bioreactor was then returned to the incubator so it could spin and wash away residual debris collected in bioreactor.

Next, the petri dish of organoids was removed from the hood and brought to the microscope to observe organoid size and imaging. Images of organoids were recorded once per week, and the approximate size of the organoids was recorded every time the bioreactor was maintained. The EVOS M5000 microscope used the 4× objective on the TRANS filter and the TRANS box checked at the bottom. The petri dish of organoids and bioreactor with DI·H₂O was brought back into the hood. The bioreactor was opened from the top, and the magnetic stir bar, baffle, and the glass rod region of the bioreactor were cleaned with the vacuum aspirator. The filter cap was removed, the DI·H₂O was poured out into a glass beaker, and the filter cap was tightly screwed on. Any remaining liquids in the bioreactor were aspirated out with the vacuum aspirator.

Next, the bioreactor was refilled with 56 mL of fresh, warm Neurobasal (NBE) media. Samples of organoids were taken out of the petri dish as needed. Using a cut 1 mL pipette tip, the remaining organoids in the petri dish and all the NBE media were placed into the bioreactor. Once the organoids reached ≥2 mm (2000 μm) in diameter, a new bioreactor was set up such that there were no more than 20 organoids in each bioreactor; when the organoids were large and solid enough, the shear stress of organoid-to-organoid collisions could affect morphology and rate of growth. It was ensured that the magnetic stir bar was evenly distributed across the baffle, then the top was tightly screwed back onto the bioreactor. It was also ensured that the stir bar and baffle were not unbalanced/lopsided or touching the dimple at the bottom of the bioreactor. The bioreactor was then replaced in the incubator on the magnetic stir plate.

Neurobasal Media Supplement Aliquot Protocol—EGF and bFGF (FGF):

TABLE 4
Recombinant Human EGF (Catalog ID: 100-26-1MG)
Recombinant Human FGF-basic 154/FGF-2 (Catalog ID: 100-146-1MG)

| | |
|---|---|
| Specifications (EGF/FGF) | Procedure (EGF/FGF) |
| Storage: At or below −20° C. | 1. Filter (0.1 - 0.2 μm pore size) 0.1% BSA |
| Shelf Life: 12 months from date of receipt when | solution to sterilize. |
| stored at −20° C. to −80° C. as supplied. | 2. Add 1 mg EGF or FGF to a final volume |
| Centrifuge vial before opening. | of 10 mL 0.1% BSA solution (100 μg/mL) |
| Suspend the product by gently pipetting the above | in appropriately labeled 15 mL centrifuge |
| recommended solution (Sterile water at 0.1 mg/mL) | tube. |
| down the sides of the vial. DO NOT VORTEX | 3. Further dilute EGF or FGF to 10 μg/mL |
| Allow several minutes for complete reconstitution. | by adding 5 mL of 100 μg/mL to 45 mL |
| For prolonged storage, dilute to working aliquots in a | of sterile filtered 0.1% BSA in |
| 0.1% bovine serum albumin (BSA) solution, store at | appropriately labeled 50 mL centrifuge |
| −20° C. and avoid repeat freeze thaws. | tube. |
| | 4. Aliquot 1 mL of 10 μg/mL EGF or FGF |
| | to appropriately labeled mini centrifuge |
| | tube. |
| | 5. Freeze tubes in −20° C. |

Calculations/Math
$Want 20 ng / mL in 500 mL media ( 10 µg total in 600 mL media ) : \frac{10 µg}{500 mL} = \frac{0.02 µg}{mL} = \frac{20 ng}{mL}$
$Given 1 mg ( 1000 µg ) EGF or FGF : \frac{1000 µg}{10 µg} = 100 tubes$
$BSA solution for stock : \frac{0.1 g BSA}{100 mL Sterile Water} = \frac{0.01 g BSA}{10 mL Sterile Water} = \frac{10 mg BSA}{10 mL Sterile Water}$
$If making both EGF and FGF stock solutions at the same time : \frac{20 mg BSA}{20 mL Sterile Water}$
$EGF or FGF stock solution : \frac{1 mg EGF or FGF}{10 mL BSA solution} = \frac{0.1 mg EGF or FGF}{1 mL BSA solution} = \frac{100 µg EGF or FGF}{1 mL BSA solution}$
${C}_{1} {V}_{1} : ( \frac{100 µg EGF or FGF}{1 mL BSA solution} ) ( {V}_{1} ) = ( \frac{10 µg EGF or FGF}{1 mL BSA solution} ) ( 1 mL )$
V₁ = 0.1 mL = 100 μL stock solution
100 μL stock solution + 900 μL BSA solution = 1 mL in a tube
If making 100 tubes of EGF or FGF:
100 tubes = 10 mL stock solution + 90 mL BSA solution
10 mL (stock solution) + 90 mL (tubes) + 5 mL (extra) = 105 mL BSA solution needed
$\frac{105 mg BSA}{105 mL Sterile Water}$
If making 50 tubes of EGF or FGF:
50 tubes = 5 mL stock solution + 45 mL BSA solution
10 mL (stock solution) + 45 mL (tubes) + 5 mL (extra) = 60 mL BSA solution needed
$\frac{60 mg BSA}{60 mL Sterile Water}$

Neurobasal Media Supplement Aliquot Protocol-Heparin: The heparin working concentration used was 8 μg/mL. First, the concentration (C1) of heparin needed per aliquot tube was determined according to:

$$8 \frac{μ g}{mL} ( 500 mL ) = ( {C}_{1} ) ( 1 mL )$$

$$4000 \frac{μ g}{mL} = 4 \frac{mg}{mL} = {C}_{1}$$

Next, the amount of heparin needed for 50 aliquots of tubes (50 mL) was determined according to:

$$4 \frac{mg}{mL} ( 50 mL ) = 200 mg Heparin$$

Next, the appropriate weight (e.g., 200 mg) of heparin was measured out and added to 40 mL of 1× phosphate buffer saline (PBS), alternatively DI·H₂O may be used, then gently mixed until the heparin was fully dissolved. The remaining volume of 1×PBS or DI·H₂O was added to reach 50 mL total. This solution was sterile filtered using a 0.1-0.2 μm pore size filter and aliquoted into appropriately labeled mini centrifuge tube (1 mL/tube).

Example #11—Spinner Flask Experiment Protocol: This protocol was used for preparation, harvesting, imaging, and sampling of the three main culture methods. The three main culture methods were the spinner flask (SF), a U-bottom shaped 96 well plate (U96), and a T-75 tissue culture flask (T-75). The constant seeding density for each culture type was 10⁶ cells per 10 mL. Sampling, imaging, and feeding occurred every five days.

Harvesting Cells in Spinner Flask: The typical cell suspension passage protocol was followed using Accutase as the dissociating solution. It was ensured that the cells were completely dissociated into single cells using the 1000 μL pipette after five minutes at 37° C. incubation in Accutase, and prior to adding neurobasal media. A viability of >90% was preferred for spinner flask culture. Ideal cell conditions (e.g., uniform sphere size, morphology, passage periods, and cell proliferation) were also preferred.

Next, a working volume was chosen for the spinner flask, using no less than 60 mL in a 100 mL spinner flask. It was preferred to have a working volume that covered the entire surface area of the spinner flask impeller and magnetic stir bar. Next, the number of cells required to match a 10⁶ cells per 10 mL media seeding density was determined. This seeding density was chosen because it reflects the typical seeding density for a T75 tissue culture flask (TCPS).

The cells were then gently added to the working volume in the spinner flask. 1 mL less than the working volume was initially added to the spinner flask. The number of cells to seed were aliquoted, spun down, and the supernatants removed. The cells were then resuspended in 1 mL of NBE media. 1 mL of cells and media was then added to the spinner flask and top cap securely fastened. The side caps were fixed to allow restricted airflow into the spinner flask.

Imaging Spinner Flask: First, DMEM/FBS media to pre-wet the pipette was warmed. The spinner flask was removed from the incubator and placed into the biological hood. The top cap of the spinner flask was removed, and the organoids were allowed 5-10 minutes to settle at the bottom. Next, a 5 mL serological pipette was pre-wet by collecting approximately 7 mL of DMEM/FBS media, holding it in the pipette for about ten seconds, then immediately replacing it back into the original bottle. Slowly and gently, a 5 mL sample of organoids was collected from the bottom of the spinner flask. This sample was placed in an appropriately sized petri-dish, which was optionally pre-wetted as well. Representative and quality images of the organoids were then taken using the compound microscope. If organoids were uniformly ≥1000 μm (1 mm), then the experiment was stopped.

Sampling Spinner Flask: Continuing from the above imaging protocol, first a centrifuge sample tube was labeled according to the intended experiment (PCR or IHC). Using a 5 mL pre-wet pipette, a 5 mL sample of the organoids was slowly collected and transferred into the 15 mL tube. The organoids were allowed 2-5 minutes to settle at the bottom. Half of the media supernatants was slowly aspirated from the tube, then approximately half of the organoid sample was collected in 1 mL. This sample was transferred into the labeled PCR mini centrifuge tube. Next, all the remaining media supernatants were aspirated from both the tubes, keeping the samples as dry as possible. The samples were not centrifuged.

SF PCR Samples: Continuing from the above sampling protocol, the samples were flash-frozen for ten seconds in liquid N₂ and then stored in the appropriately labeled sample box in the −80° C. freezer.

SF IHC Samples: Continuing from the above sampling protocol, the samples were first incubated in 200-500 μL of 4%-paraformaldehyde (4%-PF) at 4° C. overnight. After incubation, the 4%-PF was aspirated out, and the samples were washed three times with 1 mL PBS. The sample (in PBS) was then transferred from the 15 mL centrifuge tube to a new mini centrifuge tube and stored at 4° C. until ready to continue with IHC. A 1-2 mL sample was taken for PCR analysis.

Feeding Spinner Flask: The spinner flask was fed approximately every five days. The spinner flask pH was checked every day by observing the color of the phenyl red pH indicator in the NBE media. If the media was turning orange, the spinner flask was fed.

First, the organoids were allowed 5-10 minutes to settle at the bottom of the spinner flask. All but about 10 mL of supernatant media was then removed from the spinner flask. The supernatants were saved in −80° C. for future EV experiments (LC-MS and RAMAN). Next, the entire working volume of new NBE media was added to the new spinner flask, then the harvested spheres from the old spinner flask were carefully added. Finally, the new spinner flask was placed in the incubator set at 120 rpm.

Further Evaluation and Sample Requirements: Two or more spinner flask samples were required for all of these described experiments. Cell counting used a 5 mL sample with no size requirement and was conducted every five days. PCR used more than 50 spheroids having a 100-400 μm diameter, and approximately 20 spheres having a ≥400 μm diameter. IHC used as many spheroids as possible. SEM and TEM both used 20 samples. Stiffness tests used at least five samples. PCR, IHC, SEM, TEM, and stiffness tests all used spheroids at least 100 μm in diameter.

Example #12—Materials and Methods: Cell Culture: Patient-derived xenograft (PDX) GBM cell line JX6 was kindly provided by Dr. Yancey Gillespie (The University of Alabama at Birmingham). U87 and U251 were purchased from ATCC. Cells were cultured in neurobasal A medium supplemented with 1×GlutaMax (Gibco, 35050-061), 0.5×B-27 (Gibco, 12-587-010), 20 ng/ml EGF (Shenandoah, 10026), 20 ng/mL bFGF (Shenandoah, 100-146), 8 μg/mL heparin (Akron, AK3004-1000), 1× penicillin/streptomycin (Corning, 30-002-CI), and 2.5 μg/mL amphotericin B (Cytiva, SV30078.01) (NBE). Cells were seeded at a density of 1×10⁵ cells/mL in standard tissue culture flasks, U-bottom well plates (Greiner, 650970), and 100 ml bioreactors (Chemglass, CLS-1450-100) and incubated at 37° C. with 5% CO₂. GBOs were dissociated by incubating with Accutase (Corning, 25-058_CI) at 37° C. for 5 minutes followed by trituration to count cells. For continuous culture, GBOs were gently fed with 30 mL of media and passaged without dissociation for both tissue culture flask and bioreactor conditions. For GBOs in U-bottom well plate, 50% of the total volume of well was replaced with fresh NBE, with care given to not disturb the existing GBOs.

Bioreactor Culture System: Indented bottom spinner flasks with internal paddle impellers were used as small-scale bioreactors (Chemglass CL2-1450-100). The impellers and the inner surface of the bioreactors were coated using Sigmacote (Millipore Sigma, SL2) before cell inoculation. A membrane filter cap on the side of the bioreactor was used for oxygen supply. The ratio of the distance between the impeller and the baffle on the bottom was maintained at 0.15. Every 5 days, GBOs were transferred to a new bioreactor to prevent fouling. As different types of bioreactor design and impellers manipulate shear stress dynamics, two different geometries of bioreactors were used to estimate shear stress and optimize culture conditions (FIG. 5A). Given the bioreactor geometries, shear stress was estimated using the following equations (Cherry & Kwon, 1990).

$${τ}_{max} = 5 \text{.33} {{ρ}_{f} ( ϵ v )}^{\frac{1}{2}}$$

where ρ_{f} is the density of the medium and v is the kinematic viscosity of the culture medium. The viscous power dissipated per mass ϵ is determined by

$$ϵ = \frac{{N}_{P 0} {N}^{3} {D}_{a}^{5}}{{V}_{L}}$$

where N_{P0} is the dimensionless power number, N is the agitation rate, Dₐ is the impeller diameter, and V_{L} is medium volume respectively. The dimensionless power number correlates with types of impeller and baffles. The dimensionless power number was calculated by using the Kamei correlation, (Furukawa et al., 2012)

$${N}_{P 0} = \{ \frac{1.2 {π}^{4} {β}^{2}}{8 {d}^{3} ( {D}^{2} H )} \} f$$

where β is the constant value from bioreactor dimension, d is the impeller diameter, D is the bioreactor inner diameter, and H is the working volume height, and f is the friction factor respectively. All equations for each parameter are in Table 1, which shows the calculated parameters including eddies scale, impeller tip speed, and power input per volume (P/V).

High ratio of C/H (where C is the distance between the impeller and the bottom wall of the bioreactor) fails to generate homogeneous shear profiles in the bioreactor (Ismadi et al., 2014). C/H greater than 0.5 have the maximum shear stress at the wall of the bioreactor and the edges of the impellers (Ismadi et al., 2014). Thus, the C/H was maintained lower than 0.5 to have homogeneous shear profiles (Table 1).

Different dimensions of impellers not only generate various mixing profiles, but also are highly correlated with the power number (Furukawa et al., 2012). Two different ratios of the vessel diameter to the impeller diameter (D/d) were adopted to generate a high-power number (Table 2). Even with the similar estimated shear stresses, the BR2 had a higher power consumption (1.14×10⁻¹ W) than that of BR1 (9.31×10⁻⁵ W) (Table 2).

Image Analysis: Colony dimensions were measured using an automated counting macro developed in-house. Circularity and diameter of GBO were analyzed using ImageJ (Version 1.52r, NIH). Circularity was calculated using

$$Circularity = 4 π A / {p}^{2}$$

where A is the two-dimensional projected area, and p is perimeter. Images were acquired with 12-bit depth, and the raw files were analyzed using NIS-Elements Viewer (Nikon) and ImageJ (NIH). Fluorescence intensity was calculated by the corrected total cell fluorescence (CTCF).

Gene Expression Analysis: RNA was extracted without dissociation of GBOs according to the manufacturer's protocol (GeneJet RNA Purification Kit, K0731). cDNA was prepared using the kit (RevertAid First Strand cDNA synthesis Kit, K1622) and followed the manufacturer's protocol. Gene expression was quantified by qRT-PCR (StepOnePlus, Applied Biosystems) using SYBR Green (Applied Biosystems, A25742). The following genes were tested: angiogenesis (VEGFA), epithelial-mesenchymal transition (SNAI1, SNAI2, TWIST1, and TWIST2), stemness (CD133, SOX2, MET, NESTIN, and OCT4), metabolite (IDH1), hypoxia (HIF-1α, EPAS1, and ARNT), NOTCH signaling (NOTCH1, NOTCH2, NOTCH3, HES1, and HEY1), and pericyte (ACTA2). Relative expression level of genes of interest was normalized to GAPDH.

Scanning Electron Microscopy (SEM): GBOs were fixed using 2.5% glutaraldehyde in water (Millipore Sigma, G5882) for an hour at room temperature and then gradually dehydrated using ethanol (50%, 70%, 95%, and 100%) for five minutes per each concentration followed by critical point drying using CO₂ over 35° C. at 1,2000 psi. GBOs then were sputter coated with 20 nm of Au and observed by Apreo FE SEM (Thermo Scientific).

Live and Dead Cell Analysis: Cells were stained with Live and Dead Cell Kit according to manufacturer's protocol (Abcam, ab115347). Sample images were acquired and analyzed using Nikon C2 Laser Scanning Confocal Microscope.

Isolation and characterization of Extracellular Vesicles (EVs): Extracellular vesicles (EVs) were collected by sequential centrifugation: 300×g for five minutes, 1,000×g for five minutes, 4,000×g for ten minutes, 20,000×g for 20 minutes, and 100,000×g for 60 minutes. Supernatants were collected between each step. After 100,000×g centrifugation, supernatants were discarded, and the pellet was collected for image analysis. Collected EVs were transferred to the coated formvar grid. EVs were negatively stained using 2% uranyl acetate for 10 sec and washed by adding drops of deionized water, and the grids were air dried for 30 minutes. EVs images were acquired using Hitachi H-7650 Transmission Electron Microscope (TEM). Size of EVs were measured by using dynamic light scattering.

Immunohistochemistry (IHC): GBOs were fixed using 4% paraformaldehyde (Alfa Acsar, J61899AK) overnight and dehydrated using ethanol followed by xylene. Dehydrated GBOs were embedded in paraffin. Paraffin-embedded GBOs were sectioned to 5 to 10 μm thickness. Deparaffinization was done by xylene followed by ethanol. Sectioned GBOs were rehydrated with water. 10 mM sodium citrate buffer with 0.05× Tween 20 was formulated for antigen retrieval and adjusted to pH 6.0. Antigen retrieval was performed at 95° C. for ten minutes. Sectioned GBOs were stained with primary antibodies: CD133, SOX2, CD44, NOTCH1, DLL1, HIF-1α, CD31, CD34, CD144, CD146, α-SMA, NG2, GFAP, Map2, S100B, VEGFR, ZO1, and POU3f2, followed by secondary staining. Cell nuclei were counter stained by Hoechst 33342 (Invitrogen, H1399). Stained GBOs were mounted using ProLong Glass Antifade Mountant with NucBlue (Invitrogen, P36981) and imaged by Nikon C2 Laser Scanning Confocal Microscope.

Statistical Analysis: Gene expressions were statistically analyzed by Pearson correlation coefficient and one-way ANOVA using Minitab 19 and JMP Pro 15.0.0. Each gene's relative gene expression was normalized to the maximum expression level of each gene and described as ‘Z.’ Multivariate analysis was performed to analyze principal components with the default estimation method using the Correlations option. Principal component analysis (PCA) was also performed using relative gene expression of genes of interest, the size of GBO, and the agitation rates. Top two highest eigenvalues were chosen for the principal components. PCA was plotted using the coefficients in the principal components' formula. Significant difference among samples was analyzed by Tukey and Dunnett test. Statistical significance was assigned at p-value <0.05.

### DISCUSSION

In the above-disclosed examples, a small scale, matrix-free stirred tank bioreactor was employed for accelerated GBO production. Production of bGBOs with greater than 1 mm diameter was achieved within just five weeks. The bGBOs were produced with uniform sizes and without random clonal aggregations.

SEM image analysis of bGBOs showed strong cell-to-cell communications via physical interactions and secretion of EVs. EVs and their effects on cell proliferation and differentiation need to be further characterized and studied in the future. Heterogeneous size of EVs released from bGBO were observed on cell surface and highly involved in cell-to-cell interactions. These EVs can carry oncogenic EGFRvIII and their effects can vary depending on the size of EVs and culture condition (Choi et al., 2018). Thus, inhibition of cell-to-cell communication via EVs can be a potential therapeutic target.

Global gene analysis revealed that the bGBOs displayed high stemness and capability to differentiate. The gene analysis further showed that the expression of stemness genes were correlated with HIF-1α upregulation rather than with the size of the bGBOs. Upregulation of NOTCH was associated with regulating angiogenesis and differentiation. However, signaling crosstalk and other signaling pathways need to be further studied. For example, various NOTCH ligands can subsequently transduce signals and transcript multiple genes (D'Souza et al., 2008; Lino et al., 2010; Stockhausen et al., 2010; Xiu et al., 2020). It is contemplated that the heterogeneity of molecular profiles may be further elucidated by single cell RNA-sequencing analysis. A recent study demonstrated a hierarchically organized GBM roadmap by single cell RNA-sequencing and demonstrated that the cells with GSC phenotypes drive chemoresistance and GBM growth (Couturier et al., 2020). Therefore, single cell transcriptome analysis can map hierarchically organized heterogeneous bGBOs.

IHC staining revealed size-dependent and self-organized GBM TME. Development of a hypoxic core and differentiation were correlated with gene transcript analysis results. The results also showed that GBM transdifferentiated into multiple lineages and built their own GBM TME (FIG. 25, FIG. 26, FIG. 27). In particular, transdifferentiation of endothelial cells was mainly induced in the bGBOs. The large bGBOs with necrotic regions demonstrated a hierarchically organized GBM TME. The distinctive GSC niche displayed high angiogenesis, cell-to-cell interactions, and activation of transcription factors that are involved in GSC dedifferentiation and stemness. Although establishment of TME by multiple GBM cells was demonstrated, the effects of cytokines and chemokines from necrotic regions need to be further investigated. Cytokines and chemokines in GBM TME were previously shown to transform immune cells, resulting in promoting GSC survival from immune surveillance and contributing to poor prognosis (Brown et al., 2018; Zhu et al., 2012). Understanding the cell-to-cell communication via cytokines and chemokines will be critical to develop cell therapy using chimeric antigen receptor T (CAR-T) cells and regulatory T cells in the future.

In conclusion, PDX bGBO models were biomanufactured in a well-controlled manner. The bGBOs were produced from single cells, and they self-established their own GBM TME that recapitulated the heterogeneous in vivo GBM features. The GBO model can potentially be highly predictive in vitro models for preclinical study. The GBO model can also be useful for applications such as drug screening, cell therapies, and co-culture models in the future.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein or that the “particular” aspects are somehow limited in some way other than the inherent meanings of the composites and formulas literally used therein.

Exemplary aspect 1: A method of biomanufacturing organoids comprising: a) disposing a cellular starting material in a bioreactor; and b) continuously exposing the cellular starting material to mechanical stress.

Exemplary aspect 2: The method of any exemplary aspect herein, particularly example 1, wherein the cellular starting material is precursor cells, and wherein the method further includes inducing spheroid formation from precursor cells until a critical size (e.g. micrometer scale).

Exemplary aspect 3: The method of any exemplary aspect herein, particularly example 2, wherein the spheroid reaches critical threshold size when the spheroid exhibits self-differentiation.

Exemplary aspect 4: The method of any exemplary aspect herein, particularly example 3, wherein the spheroid critical size is from about 100 μm to about 600 μm.

Exemplary aspect 5: The method of any exemplary aspect herein, particularly example 1, wherein the cellular starting material is spheroids of a critical size.

Exemplary aspect 6: The method of any exemplary aspect herein, particularly example 5, wherein the spheroid reaches critical threshold size when the spheroid exhibits self-differentiation.

Exemplary aspect 7: The method of any exemplary aspect herein, particularly example 5, wherein the spheroid critical size is from about 100 μm to about 600 μm.

Exemplary aspect 8: The method of any exemplary aspect herein, particularly example 1, wherein a size of bioreactor is chosen for a desired batch size (i.e. number of product organoids).

Exemplary aspect 9: The method of any exemplary aspect herein, particularly example 1, wherein the bioreactor has a Reynolds number of about 600 to about 2200.

Exemplary aspect 10: The method of any exemplary aspect herein, particularly example 1, wherein mechanical mixing comprises shear stress and agitation.

Exemplary aspect 11: The method of any exemplary aspect herein, particularly example 10, wherein the shear stress is from about 0.1 Pa to about 0.7 Pa.

Exemplary aspect 12: The method of any exemplary aspect herein, particularly example 10, wherein a rate of agitation in the bioreactor is about 60 rpm to about 120 rpm.

Exemplary aspect 13: The method of any exemplary aspect herein, particularly example 1, wherein the cellular starting material comprises or is derived from human stem cells.

Exemplary aspect 14: The method of any exemplary aspect herein, particularly example 13, wherein the cellular starting material comprises or is derived from cancer stem cells.

Exemplary aspect 15: The method of any exemplary aspect herein, particularly example 14, wherein the cellular starting material comprises or is derived from glioblastoma stem cells or breast cancer stem cells.

Exemplary aspect 16: The method of any exemplary aspect herein, particularly example 13, wherein the cellular starting material comprises or is derived from neural progenitor cells.

Exemplary aspect 17: The method of any exemplary aspect herein, particularly example 1, wherein the organoids comprise glioblastoma organoids or breast cancer organoids.

Exemplary aspect 18: The method of any exemplary aspect herein, particularly example 1, wherein the organoids comprise healthy neural organoids.

Exemplary aspect 19: The method of any exemplary aspect herein, particularly example 1, wherein the organoids have a diameter of about 500 μm to about 5 mm.

Exemplary aspect 20: The method of any exemplary aspect herein, particularly example 1, wherein the cellular starting material is in a cell culture media formulation.

Exemplary aspect 21: The method of any exemplary aspect herein, particularly example 1, wherein cleaning-in-place protocols are used.

Exemplary aspect 22: The method of any exemplary aspect herein, particularly example 1, the method further comprises storing the organoids.

Exemplary aspect 23: A tissue model comprising an organoid, wherein the organoid is derived from human stem cells.

Exemplary aspect 24: The tissue model of any exemplary aspect herein, particularly example 23, wherein the organoid is formed without a scaffolding matrix.

Exemplary aspect 25: The tissue model of any exemplary aspect herein, particularly example 23, wherein the human stem cells comprise cancer stem cells.

Exemplary aspect 26: The tissue model of any exemplary aspect herein, particularly example 25, wherein the human stem cells comprise glioblastoma stem cells or breast cancer stem cells.

Exemplary aspect 27: The tissue model of any exemplary aspect herein, particularly example 23, wherein the human stem cells comprise neural progenitor cells.

Exemplary aspect 28: The tissue model of any exemplary aspect herein, particularly example 23, wherein the organoid comprises a hypoxic niche and a perivascular niche.

Exemplary aspect 29: The tissue model of any exemplary aspect herein, particularly example 23, wherein the organoid is stable under shear stress of about 0.1 Pa to about 0.7 Pa.

Exemplary aspect 30: The tissue model of any exemplary aspect herein, particularly example 23, wherein the organoid is stable under a rate of agitation of about 60 rpm to about 120 rpm.

Exemplary aspect 31: The tissue model of any exemplary aspect herein, particularly example 23, wherein the organoid has a diameter of from about 500 μm to about 5 mm.

Exemplary aspect 32: The tissue model of any exemplary aspect herein, particularly example 23, further comprising axon-like protrusions.

Exemplary aspect 33: The tissue model of any exemplary aspect herein, particularly example 23, wherein the organoid comprises a glioblastoma organoid or a breast cancer organoid.

Exemplary aspect 34: The tissue model of any exemplary aspect herein, particularly example 23, wherein the organoid comprises a healthy neural organoid.

The following patents, applications and publications as listed below and throughout this document are hereby incorporated by reference in their entirety herein.

### REFERENCES

ADDIN EN.REFLIST Ahn, S. H., Park, H., Ahn, Y. H., Kim, S., Cho, M. S., Kang. J. L., & Choi, Y. H. (2016). Necrotic cells influence migration and invasion of glioblastoma via NF-kappa B/AP-1-mediated IL-8 regulation. Scientific Reports, 6.
Barisam, M., Saidi, M. S., Kashaninejad, N., & Nguyen, N. T. (2018). Prediction of Necrotic Core and Hypoxic Zone of Multicellular Spheroids in a Microbioreactor with a U-Shaped Barrier. Micromachines, 9 (3).
Bayin, N. S., Frenster, J. D., Sen, R., Si, S., Modrek, A. S., Galifianakis, N. Placantonakis, D. G. (2017). Notch signaling regulates metabolic heterogeneity in glioblastoma stem cells. Oncotarget, 8 (39), 64932-64953.
Beasley, N. J. P., Wykoff, C. C., Watson, P. H., Leek, R., Turley, H., Gatter, K., Harris, A. L. (2001). Carbonic anhydrase IX, an endogenous hypoxia marker, expression in head and neck squamous cell carcinoma and its relationship to hypoxia, necrosis, and microvessel density. Cancer Res. 61 (13). 5262-5267.

Benedito. R., Roca. C., Sorensen, I. Adams, S., Gossler, A., Fruttiger, M., & Adams. R. H. (2009). The Notch Ligands DI14 and Jagged1 Have Opposing Effects on Angiogenesis. Cell. 137 (6), 1124-1135.
Bhat. A. A., Uppada, S., Achkar, I. W., Hashem. S., Yadav. S. K., Shanmugakonar, M. Uddin. S. (2019). Tight Junction Proteins and Signaling Pathways in Cancer and Inflammation: A Functional Crosstalk. Front Physiol. 9.
Brown. D. V. Filiz. G., Daniel. P. M., Hollande, F., Dworkin, S., Amiridis, S. Mantamadiotis. T. (2017). Expression of CD133 and CD44 in glioblastoma stem cells correlates with cell proliferation, phenotype stability and intra-tumor heterogeneity. PLOS One, 12 (2).
Brown. N. F., Carter. T. J., Ottaviani. D., & Mulholland. P. (2018). Harnessing the immune system in glioblastoma. British Journal of Cancer. 119 (10). 1171-1181.
Charles. N., Ozawa. T., Squatrito, M., Bleau, A. M., Brennan, C. W., Hambardzumyan, D., & Holland, E. C. (2010). Perivascular Nitric Oxide Activates Notch Signaling and Promotes Stem-like Character in PDGF-Induced Glioma Cells. Cell Stem Cell. 6 (2). 141-152.
Cherry. R. S., & Kwon. K. Y. (1990). Transient Shear Stresses on a Suspension Cell in Turbulence. Biotechnol Bioeng. 36 (6). 563-571.
Choi. D., Montermini, L., Kim. D. K., Mechan. B., Roth. F. P., & Rak. J. (2018). The Impact of Oncogenic EGFRvIII on the Proteome of Extracellular Vesicles Released from Glioblastoma Cells. Molecular & Cellular Proteomics, 17 (10). 1948-1964.
Christensen, K. Schroder. H. D., & Kristensen. B. W. (2011). CD133 (+) niches and single cells in glioblastoma have different phenotypes. J Neurooncol, 104 (1). 129-143.
Couturier, C. P., Ayyadhury, S., Le. P. U., Nadaf. J., Monlong. J., Riva, G. Petrecca, K. (2020). Single-cell RNA-seq reveals that glioblastoma recapitulates a normal neurodevelopmental hierarchy. Nature Communications, 11 (1).
D'Souza, B., Miyamoto. A., & Weinmaster. G. (2008). The many facets of Notch ligands. Oncogene. 27 (38). 5148-5167.
Daster. S., Amatruda. N. Calabrese. D., Ivanck. R., Turrini. E., Drocser. R. A. Muraro, M. G. (2017). Induction of hypoxia and necrosis in multicellular tumor spheroids is associated with resistance to chemotherapy treatment. Oncotarget, 8 (1). 1725-1736.
Desai. A. Glaser. A., Liu. D. L. Raghavachari. N., Blum. A., Zalos. G. Cannon. R. O. (2009). Microarray-Based Characterization of a Colony Assay Used to Investigate Endothelial Progenitor Cells and Relevance to Endothelial Function in Humans. Arteriosclerosis Thrombosis and Vascular Biology. 29 (1). 121-127.
Desmouliere. A. Geinoz. A., Gabbiani. F. & Gabbiani, G. (1993). Transforming Growth-Factor-Beta-1 Induces Alpha-Smooth Muscle Actin Expression in Granulation-Tissue Myofibroblasts and in Quiescent and Growing Cultured Fibroblasts. Journal of Cell Biology, 122 (1), 103-111.
Dong. Z., Zhang. G. X., Qu. M., Gimple, R. C., Wu, Q. L., Qiu. Z. X. Rich, J. N. (2019). Targeting Glioblastoma Stem Cells through Disruption of the Circadian Clock. Cancer Discovery, 9 (11). 1556-1573.
Emlet. D. R., Gupta. P., Holgado-Madruga, M., Del Vecchio, C. A., Mitra, S. S., Han. S. Y. Wong. A. J. (2014). Targeting a Glioblastoma Cancer Stem-Cell Population Defined by EGF Receptor Variant III. Cancer Res. 74 (4). 1238-1249.
Fan. X. Khaki. L., Zhu. T. S. Soules. M. E., Talsma. C. E., Gul. N. Eberhart. C. G. (2010). NOTCH Pathway Blockade Depletes CD133-Positive Glioblastoma Cells and Inhibits Growth of Tumor Neurospheres and Xenografts. Stem Cells, 28 (1). 5-16.
Frank. N. Y., Schatton. T., Kim. S., Zhan, Q. A., Wilson, B. J., Ma. J. Frank, M. H. (2011). VEGFR-1 Expressed by Malignant Melanoma-Initiating Cells Is Required for Tumor Growth. Cancer Res. 71 (4). 1474-1485.
Furukawa. H., Kato. Y., Inouc. Y., Kato, T., Tada, Y. & Hashimoto. S. (2012). Correlation of Power Consumption for Several Kinds of Mixing Impellers. International Journal of Chemical Engineering. 2012, 1-6.
He. H., Niu. C. S. & Li. M. W. (2012). Correlation between glioblastoma stem-like cells and tumor vascularization. Oncology Reports, 27 (1). 45-50.
Hellstrom. M., Phng. L. K. Hofmann. J. J., Wallgard. E., Coultas. L., Lindblom, P. Betsholtz, C. (2007). DI14 signalling through Notch1 regulates formation of tip cells during angiogenesis. Nature. 445 (7129). 776-780.
Hilbe. W. Dirnhofer. S., Oberwasserlechner. F., Schmid. T., Gunsilius, E., Hilbe, G. Kahler. C. M. (2004). CD133 positive endothelial progenitor cells contribute to the tumour vasculature in non-small cell lung cancer. Journal of Clinical Pathology. 57 (9). 965-969.
Hristov. M., Erl. W., & Weber. P. C. (2003). Endothelial progenitor cells-Mobilization, differentiation, and homing. Arteriosclerosis Thrombosis and Vascular Biology, 23 (7). 1185-1189.
Hu. B. Wu. Z., & Phan. S. H. (2003). Smad3 mediates transforming growth factor-beta-induced alpha-smooth muscle actin expression. American Journal of Respiratory Cell and Molecular Biology. 29 (3). 397-404.
Hubert. C. G., Rivera, M. Spangler, L. C., Wu, Q. L., Mack, S. C., Prager, B. C. Rich, J. N. (2016). A Three-Dimensional Organoid Culture System Derived from Human Glioblastomas Recapitulates the Hypoxic Gradients and Cancer Stem Cell Heterogeneity of Tumors Found In vivo. Cancer Res. 76 (8). 2465-2477.
Ismadi. M. Z., Gupta. P., Fouras, A., Verma, P., Jadhav. S., Bellare, J., & Hourigan, K. (2014). Flow Characterization of a Spinner Flask for Induced Pluripotent Stem Cell Culture Application. PLOS One. 9 (10).
Jacob. F., Salinas. R. D., Zhang. D. Y., Nguyen. P. T. T., Schnoll. J. G., Wong. S. Z. H. Song. H. J. (2020). A Patient-Derived Glioblastoma Organoid Model and Biobank Recapitulates Inter- and Intra-tumoral Heterogencity. Cell. 180 (1). 188-+.
Kaihara. T., Kawamata, H., Imura, J., Fujii. S., Kitajima, K., Omotchara. F. Fujimori, T. (2003). Redifferentiation and ZO-1 reexpression in liver-metastasized colorectal cancer: Possible association with epidermal growth factor receptor-induced tyrosine phosphorylation of ZO-1. Cancer Science. 94 (2). 166-172.
Klein. E., Hau. A.-C., Oudin. A. Golebicwska. A., & Niclou. S. P. (2020). Glioblastoma Organoids: Pre-Clinical Applications and Challenges in the Context of Immunotherapy. Front Oncol, 10.
Lancaster. M. A., Renner. M., Martin, C. A., Wenzel, D., Bicknell, L. S., Hurles, M. E. Knoblich. J. A. (2013). Cerebral organoids model human brain development and microcephaly. Nature. 501 (7467). 373-+.
Lathia. J. D., Mack. S. C., Mulkearns-Hubert. E. E., Valentim, C. L. L., & Rich. J. N. (2015). Cancer stem cells in glioblastoma. Genes & Development. 29 (12), 1203-1217.
Lec. G., Auffinger. B., Guo, D. N., Hasan. T., Deheeger. M., Tobias, A. L. Ahmed. A. U. (2016). Dedifferentiation of Glioma Cells to Glioma Stem-like Cells By Therapeutic Stress-induced HIF Signaling in the Recurrent GBM Model. Molecular Cancer Therapeutics, 15 (12). 3064-3076.
Lee. J., Kotliarova, S., Kotliarov, Y., Li. A. G., Su, Q., Donin, N. M. Finc. H. A. (2006). Tumor stem cells derived from glioblastomas cultured in bFGF and EGF more closely mirror the phenotype and genotype of primary tumors than do serum-cultured cell lines. Cancer Cell. 9 (5). 391-403.
Li. J. L. Sainson. R. C. A., Oon, C. E., Turley. H., Leck. R., Sheldon, H. Harris, A. L. (2011). DLL4-Notch Signaling Mediates Tumor Resistance to Anti-VEGF Therapy In vivo. Cancer Res. 71 (18). 6073-6083.
Lino. M. M. Merlo. A., & Boulay. J. L. (2010). Notch signaling in glioblastoma: a developmental drug target? Bmc Medicine. 8.
Liu. N., Zang. R., Yang. S.-T., & Li. Y. (2014). Stem cell engineering in bioreactors for large-scale bioprocessing. Engineering in Life Sciences, 14 (1), 4-15.
Liu. S., Wang. Y. Y., Xu. K. B., Wang. Z., Fan. X., Zhang. C. B. Jiang. T. (2017). Relationship between necrotic patterns in glioblastoma and patient survival: fractal dimension and lacunarity analyses using magnetic resonance imaging. Scientific Reports. 7.
Luo, P. L., Wang. Y. J., Yang. Y. Y., & Yang. J. J. (2018). Hypoxia-induced hyperpermeability of rat glomerular endothelial cells involves HIF-2 alpha mediated changes in the expression of occludin and ZO-1. Brazilian Journal of Medical and Biological Research. 51 (7).
Matter. K., Aijaz, S., Tsapara, A., & Balda, M. S. (2005). Mammalian tight junctions in the regulation of epithelial differentiation and proliferation. Current Opinion in Cell Biology. 17 (5). 453-458.
Nakano. I., Garnier. D., Minata. M., & Rak. J. (2015). Extracellular vesicles in the biology of brain tumour stem cells—Implications for inter-cellular communication, therapy and biomarker development. Semin Cell Dev Biol. 40, 17-26.
Ogawa, J., Pao. G. M., Shokhirev. M. N. & Verma. I. M. (2018). Glioblastoma Model Using Human Cerebral Organoids. Cell Reports, 23 (4), 1220-1229.
Ostrom, Q. T., Patil, N., Cioffi, G., Waite. K., Kruchko, C., & Barnholtz-Sloan. J. S. (2020). CBTRUS Statistical Report: Primary Brain and Other Central Nervous System Tumors Diagnosed in the United States in 2013-2017. Neuro Oncol. 22, 1-42.
Panchalingam, K. M., Paramchuk. W. J., Chiang. C. Y., Shah. N., Madan. A., Hood. L. Behie. L. A. (2010). Bioprocessing of human glioblastoma brain cancer tissue. Tissue Eng Part A. 16 (4), 1169-1177.
Park. K. S. Kang. S. N., Kim, D. H., Kim. H. B., Im. K. S., Park. W. Joung. Y. K. (2020). Late endothelial progenitor cell-capture stents with CD146 antibody and nanostructure reduce in-stent restenosis and thrombosis. Acta Biomaterialia, 111, 91-101.
Patrizii. M., Bartucci, M., Pinc. S. R. & Sabaawy, H. E. (2018). Utility of Glioblastoma Patient-Derived Orthotopic Xenografts in Drug Discovery and Personalized therapy. Front Oncol. 8.
Ricci-Vitiani. L., Pallini. R., Biffoni, M., Todaro, M., Invernici. G. Cenci. T. De Maria, R. (2011). Tumour vascularization via endothelial differentiation of glioblastoma stem-like cells (vol 468, pg 824, 2010). Nature. 477 (7363). 238-238.
Rocha. R., Torres. A., Ojeda. K., Uribe. D., Rocha, D., Erices. J. Quezada. C. (2018). The Adenosine A (3) Receptor Regulates Differentiation of Glioblastoma Stem-Like Cells to Endothelial Cells under Hypoxia. International Journal of Molecular Sciences, 19 (4).
Rong. Y., Durden, D. L., Van Meir. E. G., & Brat. D. J. (2006). ‘Pseudopalisading’ necrosis in glioblastoma: A familiar morphologic feature that links vascular pathology, hypoxia, and angiogenesis. J Neuropathol Exp Neurol. 65 (6). 529-539.
Shah. S. S., Rodriguez, G. A., Musick. A., Walters. W. M., De Cordoba, N., Barbarite, E. Graham. R. M. (2019). Targeting Glioblastoma Stem Cells with 2-Deoxy-D-Glucose (2-DG) Potentiates Radiation-Induced Unfolded Protein Response (UPR). Cancers, 11 (2).
Sherry, M. M., Reeves. A., Wu. J. L. K., & Cochran, B. H. (2009). STAT3 Is Required for Proliferation and Maintenance of Multipotency in Glioblastoma Stem Cells. Stem Cells, 27 (10). 2383-2392.
Stockhausen. M. T., Kristoffersen. K., & Poulsen. H. S. (2010). The functional role of Notch signaling in human gliomas. Neuro Oncol, 12 (2), 199-211.
Suva. M. L., Rheinbay. E., Gillespic. S. M., Patel, A. P., Wakimoto, H., Rabkin. S. D. Bernstein, B. E. (2014). Reconstructing and Reprogramming the Tumor-Propagating Potential of Glioblastoma Stem-like Cells. Cell. 157 (3). 580-594.
Tilson. S. G., Haley. E. M., Triantafillu. U. L., Dozier, D. A., Langford, C. P., Gillespie, G. Y., & Kim, Y. (2015). ROCK Inhibition Facilitates In vitro Expansion of Glioblastoma Stem-Like Cells. PLOS One. 10 (7). e0132823.
Timmermans. F., Plum. J., Yoder. M. C., Ingram, D. A., Vandekerckhove. B., & Casc. J. (2009). Endothelial progenitor cells: identity defined? Journal of Cellular and Molecular Medicine, 13 (1), 87-102.
van Linde. M. E., Brahm. C. G., Hamer. P. C. D., Reijneveld. J. C., Bruynzeel. A. M. E., Vandertop. W. P. Verheul. H. M. W. (2017). Treatment outcome of patients with recurrent glioblastoma multiforme: a retrospective multicenter analysis. J Neurooncol, 135 (1). 183-192.
Vora, P., Venugopal, C., Salim, S. K., Tatari, N., Bakhshinyan, D., Singh, M., Singh, S. (2020). The Rational Development of CD133-Targeting Immunotherapies for Glioblastoma. Cell Stem Cell, 26 (6), 832-+.
Wang, X. X., Prager, B. C., Wu, Q. L., Kim, L. J. Y., Gimple, R. C., Shi, Y., Rich, J. N. (2018). Reciprocal Signaling between Glioblastoma Stem Cells and Differentiated Tumor Cells Promotes Malignant Progression. Cell Stem Cell, 22 (4), 514-+.
Warshamana, G. S., Corti, M., & Brody, A. R. (2001). TNF-alpha, PDGF, and TGF-beta (1) expression by primary mouse bronchiolar-alveolar epithelial and mesenchymal cells: TNF-alpha induces TGF-beta (1). Experimental and Molecular Pathology, 71 (1), 13-33.
Xing, T. S., Benderman, L. L. A. M. N., Sabu, T. Y., Parker, O. E., Yang, E. R., Lu, Q., Chen, Y. H. (2020). Tight Junction Protein Claudin-7 Is Essential for Intestinal Epithelial Stem Cell Self-Renewal and Differentiation. Cellular and Molecular Gastroenterology and Hepatology, 9 (4), 641-659.
Xiu, M. X., Liu, Y. M., & Kuang, B. H. (2020). The oncogenic role of Jagged1/Notch signaling in cancer. Biomedicine & Pharmacotherapy, 129.
Xu, J. L., Lim, S. B. H., Ng, M. Y., Ali, S. M., Kausalya, J. P., Limviphuvadh, V., Hunziker, W. (2012). ZO-1 Regulates Erk, Smad1/5/8, Smad2, and RhoA Activities to Modulate Self-Renewal and Differentiation of Mouse Embryonic Stem Cells. Stem Cells, 30 (9), 1885-1900.
Yoshimatsu, Y., Wakabayashi, I., Kimuro, S., Takahashi, N., Takahashi, K., Kobayashi, M., Watabe, T. (2020). TNF-α enhances TGF-β-induced endothelial-to-mesenchymal transition via TGF-β signal augmentation. Cancer Science, 111 (7), 2385-2399.
Zhu, T. S., Costello, M. A., Talsma, C. E., Flack, C. G., Crowley, J. G., Hamm, L. L., Fan, X. (2011). Endothelial Cells Create a Stem Cell Niche in Glioblastoma by Providing NOTCH Ligands That Nurture Self-Renewal of Cancer Stem-Like Cells. Cancer Res, 71 (18), 6061-6072.
Zhu, V. F., Yang, J. X., LeBrun, D. G., & Li, M. (2012). Understanding the role of cytokines in Glioblastoma Multiforme pathogenesis. Cancer Lett, 316 (2), 139-150.
Sundar, S. J., Shakya, S., Barnett, A., Wallace, L. C., Jeon, H., Sloan, A., et al. (2022). Three-dimensional organoid culture unveils resistance to clinical therapies in adult and pediatric glioblastoma. Translational Oncology, 15 (1), 101251.

What is claimed is:

1. A method of biomanufacturing organoids comprising:
a) disposing a cellular starting material in a bioreactor; and
b) continuously exposing the cellular starting material to mechanical stress.

2. The method of claim 1, wherein the cellular starting material is precursor cells, and wherein the method further includes inducing spheroid formation from precursor cells until a critical size.

3. The method of claim 2, wherein the spheroid reaches critical threshold size when the spheroid exhibits self-differentiation, and wherein the spheroid critical size is from about 100 μm to about 600 μm.

4. The method of claim 1, wherein the cellular starting material is spheroids of a critical size.

5. The method of claim 4, wherein the spheroid reaches critical threshold size when the spheroid exhibits self-differentiation, and wherein the spheroid critical size is from about 100 μm to about 600 μm.

6. The method of claim 1, wherein the bioreactor has a Reynolds number of about 600 to about 2200.

7. The method of claim 1, wherein mechanical mixing comprises shear stress and agitation, wherein the shear stress is from about 0.1 Pa to about 0.7 Pa, and wherein a rate of agitation in the bioreactor is about 60 rpm to about 120 rpm.

8. The method of claim 1, wherein the cellular starting material comprises or is derived from human stem cells.

9. The method of claim 8, wherein the cellular starting material comprises or is derived from cancer stem cells or neural progenitor cells.

10. The method of claim 9, wherein the cellular starting material comprises or is derived from glioblastoma stem cells or breast cancer stem cells.

11. The method of claim 1, wherein the organoids comprise glioblastoma organoids, breast cancer organoids, or healthy neural organoids.

12. The method of claim 1, wherein the organoids have a diameter of about 500 μm to about 5 mm.

13. A tissue model comprising an organoid, wherein the organoid is derived from human stem cells.

14. The tissue model of claim 13, wherein the organoid is formed without a scaffolding matrix.

15. The tissue model of claim 13, wherein the human stem cells comprise cancer stem cells or neural progenitor cells.

16. The tissue model of claim 15, wherein the human stem cells comprise glioblastoma stem cells or breast cancer stem cells.

17. The tissue model of claim 13, wherein the organoid comprises a hypoxic niche and a perivascular niche and/or axon-like protrusions.

18. The tissue model of claim 13, wherein the organoid is stable under shear stress of about 0.1 Pa to about 0.7 Pa and/or a rate of agitation of about 60 rpm to about 120 rpm.

19. The tissue model of claim 13, wherein the organoid has a diameter of from about 500 μm to about 5 mm.

20. The tissue model of claim 13, wherein the organoid comprises a glioblastoma organoid, a breast cancer organoid, or a healthy neural organoid.
